(19) United States
(12) Patent Application Publication
(10) Pub. No.: US 2024/0120067 A1
(43) Pub. Date: Apr. 11, 2024

(54) ARTIFICIAL INTELLIGENCE METHOD FOR DETERMINING THERAPY RECOMENDATION FOR INDIVIDUALS WITH NEURODEVELOPMENTAL DISORDERS

(71) Applicant: Montera d/b/a Forta, San Francisco, CA (US)

(72) Inventors: Jenish MAHARJAN, San Francisco, CA (US); Anurag GARIKIPATI, San Francisco, CA (US); Madalina CIOBANU, San Francisco, CA (US); Frank DINENNO, San Francisco, CA (US); Qingqing MAO, San Francisco, CA (US)

(21) Appl. No.: 17/955,616

(22) Filed: Sep. 29, 2022

(51) Int. Cl. G16H 20/70; G16H 50/20

(52) U.S. Cl. CPC G16H 20/70; G16H 50/20

(57) ABSTRACT

A method implemented via a computing device. The method may include receiving, by the computing device, data associated with a subject having a neurodevelopmental disorder (NDD). The data associated with the subject may include demographic data, schooling data, family medical data, prior therapy data, observational assessment data, medication data, goals data, or combinations thereof. The method may further include evaluating, by the computing device, the data associated with the subject via a neurodevelopmental disorder treatment recommendation (NDDTR) model. The NDDTR model may evaluate the data associated with the subject to determine a therapy recommendation. The therapy recommendation may include a standard of care. The NDD may be ASD.

### TECHNICAL FIELD

The present disclosure relates to methods and systems pertaining to the use of artificial intelligence for the determining and providing a therapy recommendation for individuals with a neurodevelopmental disorder (NDD) such as autism spectrum disorder (ASD).

### BACKGROUND

Neurodevelopmental disorders are generally associated with impaired neurological development, often leading to abnormal brain function, and exhibited as emotional, learning, behavioral, and/or cognitive aberrances that can affect sensory systems, motor systems, speech, and language. Some examples of neurodevelopmental disorders include ASD, attention deficit hyperactivity disorder (ADHD), cerebral palsy, Rett syndrome, and Tourette's syndrome.

ASD is a complex neurodevelopmental disorder which expresses heterogeneously in afflicted individuals, although a few essential features are commonly present: social communication impairment as well as restricted interests and repetitive behaviors. It is estimated that currently about 1 in 100 children worldwide are diagnosed with ASD, while the Centers for Disease Control and Prevention (CDC) estimates based on 2018 data that about 1 in 44 8-year-old children have been identified with ASD in the United States. ASD occurs across all geographic regions and socio-economic groups.

Conventionally, determination of a therapy recommendation for an individual (e.g., a person, a patient, a subject, etc.) having an NDD, such as ASD, is a challenging, elaborate, and time-intensive process including consideration of various factors by a clinician or other healthcare professionals. For instance, determination of a therapy recommendation that will be effective is often difficult because of the nature of NDDs and because NDDs often present with other comorbidities, which can be of a neurodevelopmental or other medical nature.

Generally, the likelihood of receiving treatment for an NDD relatively earlier in life is associated with a relatively higher socio-economic status of the family, and individuals having a relatively lower socio-economic status tend to receive therapy at a relatively later age. Generally, individuals that live in rural and other underserved communities often receive treatment at lower rates. Also, earlier treatment of individuals having an NDD, such as ASD, may be associated with better prognosis, for example, a better quality of life, ranging from significant gains in cognition, language, and adaptive behavior to more functional outcomes in later life. Individuals with ASD that do not receive early intervention have a higher degree of difficulty conveying their symptoms (owing to a language deficit), while tending to exhibit disruptive behaviors. This, in turn, may mask other neurodevelopmental and/or medical conditions which may remain undiagnosed, thus causing both short-term as well as long-term problems for the undiagnosed individual.

Given the complex and challenging nature of determining therapy recommendations for individuals having an NDD, such as ASD, there is an ongoing need to develop and provide therapy recommendations to these individuals and, at the same time, to provide easier, more readily-available access to such methods.

### BRIEF SUMMARY

Disclosed herein is a method implemented via a computing device. The method may comprise receiving, by the computing device, data associated with a subject having a neurodevelopmental disorder (NDD). The data associated with the subject may comprise demographic data, schooling data, family medical data, prior therapy data, observational assessment data, medication data, goals data, or combinations thereof. The method may also comprise evaluating, by the computing device, the data associated with the subject via a neurodevelopmental disorder treatment recommendation (NDDTR) model, wherein the NDDTR model is configured to evaluate the data associated with the subject to determine a therapy recommendation, wherein the therapy recommendation comprises a standard of care.

Additionally or alternatively, also disclosed herein is a computing system. The system may comprise a computing device. The computing device may comprise a processor and a non-transitory computer-readable medium. The non-transitory computer-readable medium may include instructions configured to cause the processor to implement an NDDTR model. The NDDTR model, when implemented via the processor, may cause the computing device to receive data associated with a subject having an NDD. The data associated with the subject may comprise demographic data, schooling data, family medical data, prior therapy data, observational assessment data, medication data, goals data, or combinations thereof. The NDDTR model, when implemented via the processor, may also cause the computing device to evaluate the data associated with the subject via an NDDTR model. The NDDTR model may be configured to evaluate the data associated with the subject to determine a therapy recommendation. The therapy recommendation may comprise a standard of care.

Additionally or alternatively, also disclosed herein is a method implemented via a computing device. The method may comprise receiving, by the computing device, training data associated with a plurality of subjects, wherein at least a portion of the subjects are individuals characterized as having an NDD. The training data associated with each of the plurality of subjects may comprise demographic data, schooling data, family medical data, prior therapy data, observational assessment data, medication data, goals data, or combinations thereof. The method may also comprise processing the training data associated with the plurality of subjects to yield an NDDTR model. The NDDTR model may be configured to evaluate data associated with a subject to evaluate the data associated with the subject to determine a therapy recommendation. The therapy recommendation may comprise a standard of care.

### BRIEF DESCRIPTION OF THE DRAWINGS

For a detailed description of the preferred aspects of the disclosed processes and systems, reference will now be made to the accompanying drawings in which:

FIG. 1 displays a schematic diagram of an embodiment of the implementation of a model as disclosed herein;

FIG. 2 displays a schematic diagram of an additional or alternative embodiment of the implementation of a model as disclosed herein;

FIG. 3 is a schematic representation of a computing system by way of which a machine learning model may be employed;

FIG. 4 is a schematic representation of a machine learning model;

FIG. 5 is a schematic diagram of an embodiment of methods related to a model as disclosed herein;

FIG. 6 is a diagram of certain results related to an embodiment of a model of the type disclosed herein;

FIG. 7 is a diagram of certain results related to another embodiment of a model of the type disclosed herein;

FIG. 8 is a diagram of certain results related to another embodiment of a model of the type disclosed herein;

FIG. 9 is a diagram of certain results related to an embodiment of a model of the type disclosed herein;

FIG. 10 are diagrams of certain results related to an embodiment of a model of the type disclosed herein;

FIG. 11 is a diagram of certain results related to an embodiment of a model of the type disclosed herein;

FIG. 12 is a diagram of certain results related to an embodiment of a model of the type disclosed herein; and

FIG. 13 is a diagram of certain results related to an embodiment of a model of the type disclosed herein.

### DETAILED DESCRIPTION

In various embodiments disclosed herein are methods, systems, and devices related to the determination and provision of a therapy recommendation for individuals having a neurodevelopmental disorder (NDD). Examples of an NDD for which a therapy recommendation may be determined and provided include disorders on the autism spectrum or autism spectrum disorder (ASD); attention deficit hyperactivity disorder (ADHD), other specified ADHD, unspecified ADHD; motor disorders, developmental coordination disorder, stereotypic movement disorder, tic disorders, Tourette's disorder or syndrome, persistent (chronic) motor or vocal tic disorder, provisional tic disorder, other specified tic disorder, unspecified tic disorder; cerebral palsy; Rett syndrome; intellectual disabilities, intellectual developmental disorder, global developmental delay, unspecified intellectual disability, unspecified intellectual developmental disorder; communication disorders, language disorder, speech sound disorder or phonological disorder, childhood-onset fluency disorder or stuttering; social or pragmatic communication disorder, unspecified communication disorder; specific learning disorder; other NDDs, other specified NDD, and unspecified NDD.

In an aspect, the NDD comprises ASD. For purposes of the disclosure herein, the terms “disorder on the autism spectrum” and “ASD” may be used interchangeably to refer to a disorder encompassing autistic disorder, Asperger's disorder, pervasive developmental disorder—not otherwise specified (PDD-NOS), or ASD, where such disorders meet the diagnostic criteria of an accepted or recognized standard for diagnosis of the relevant disorder, for example, the Diagnostic and Statistical Manual of Mental Disorders, 4th Edition (DSM-IV), the Diagnostic and Statistical Manual of Mental Disorders, Fifth Edition (DSM-5), both the DSM-IV and DSM-5, or a later iteration thereof. In some embodiments, the disclosed methods, systems, and devices may be effective to determine and provide for a therapy recommendation for individuals having the ASD, for example, an individual having an ASD subtype, such as autistic disorder, Asperger's disorder, PDD-NOS, or some alternative or additional subclassification. Generally, the terms “subject” and “patient” may be used interchangeably to refer to an individual, that is, a human, pertinent to one or more aspects of the disclosed subject matter. Further, the terms “therapy” and “treatment” may be used interchangeably to refer to services (e.g., therapeutical services, medical services, intervention services, etc.) delivered to a particular individual with the purpose of improving or alleviating NDD symptoms for that particular individual, wherein such services are pertinent to one or more aspects of the disclosed subject matter.

The methods, systems, and devices disclosed herein may be effective or function to provide for a treatment recommendation for an individual having ASD. The ASD may be officially diagnosed by a health care professional, alternatively, provisionally diagnosed by a health care professional, or alternatively, not diagnosed by a health care professional. As would be appreciated by one of skill in the art with the help of this disclosure, provisional diagnoses of ASD can be delivered by a health care professional. Further, and as would be appreciated by one of skill in the art with the help of this disclosure, self-diagnosis of ASD (e.g., not diagnosed by a health care professional) is generally accepted in the ASD community at large. Furthermore, and as would be appreciated by one of skill in the art with the help of this disclosure, individuals lacking a diagnosis of ASD may present symptoms associated with ASD and therefore may benefit from therapy or a therapy recommendation that is usually delivered for individuals that hold an official ASD diagnosis.

In some embodiments, the disclosed methods, systems, and devices may implement a model effective to determine and provide a therapy recommendation, for example, a neurodevelopmental disorder treatment recommendation (NDDTR) model. Generally, the NDDTR model may be configured to evaluate the data associated with the subject to determine a therapy recommendation, for example, to output a standard of care.

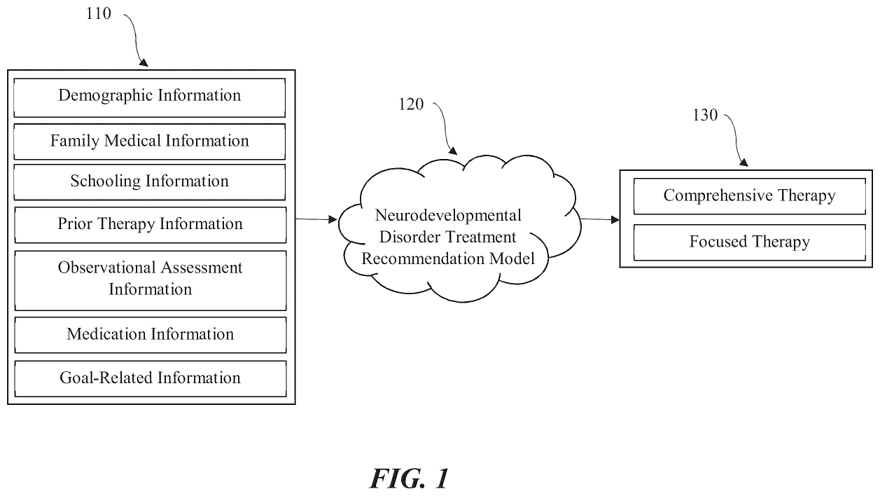

Referring to FIG. 1, an embodiment of the implementation of a model, for example, the NDDTR model 120 illustrated. For example, in the embodiment of FIG. 1, data associated with the subject, as will be disclosed herein, is utilized as inputs 110 by an NDDTR model 120. In the embodiment of FIG. 1, the subject may be characterized as having been previously identified as having an NDD, for example, ASD.

As will be discussed herein, the NDDTR model 120 may be configured to evaluate the data associated with the subject to determine a therapy recommendation. For example, evaluation of the data associated with the subject by the NDDTR model 120 may yield a therapy recommendation that includes a standard of care 130. In various embodiments, the standard of care may include an indication of the intensity of therapy for the subject, an indication of services for the subject, an indication of one of a comprehensive therapy or a focused therapy. As used herein, the term “comprehensive therapy” refers to a therapy regimen that is intended to treat multiple developmental domains as a part of the therapy regimen, for example, cognitive, communicative, social, emotional, and/or adaptive domains. Generally, although not necessarily, a comprehensive therapy may be associated with a treatment intensity of about 25 to about 40 hours per week of direct treatment to the subject. Also, as used herein, the term “focused therapy” refers to a therapy regimen that is intended to treat a limited number of developmental domains. Generally, although not necessarily, a focused therapy may be associated with a treatment intensity of about 10 to about 25 hours per week of direct treatment to the subject. The standard of care may further comprise one or more additional aspects, such as additional supervision or a training program for a caregiver.

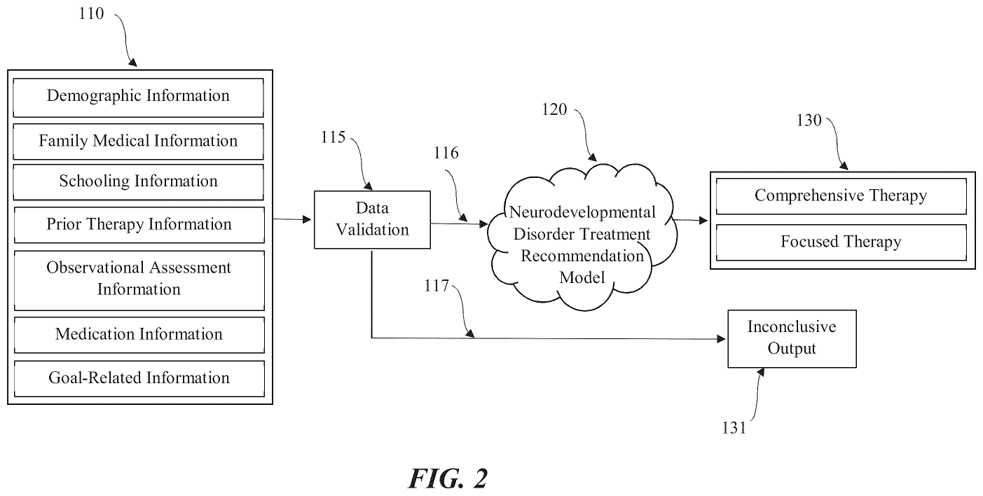

Referring to FIG. 2, in some embodiments, the NDDTR model 120 may be configured to evaluate previously validated data. For example, in some embodiments the inputs may be subjected to validation 115. In some embodiments, the validation 115 of the data associated with the subject may separate valid data 116 from any other data such that only valid data 116 is then input into the NDDTR model 120. Additionally or alternatively, as an example, validation 115 of the data associated with the subject may also separate invalid data 117 from any other data such that the invalid data 117 is not input into the NDDTR model 120, which could have the effect of leading to an errant or inconclusive output 131. For purposes of the disclosure herein, the term “valid data” refers to data that can be evaluated by the NDDTR model 120 to determine and provide the therapy recommendation, for example, according to the disclosure that follows. Further, and for purposes of the disclosure herein, the term “invalid data” refers to data that, if processed by the NDDTR model 120, may lead to an inconclusive, incorrect, or illogical result. For example, invalid data containing significant outliers may include data which are greater than about 1 standard deviation away from the mean of the entire dataset, alternatively greater than about 1.5 standard deviations away from the mean of the entire dataset, alternatively greater than about 2 standard deviations away from the mean of the entire dataset, or alternatively greater than about 1.5 times the interquartile range of the entire dataset. The invalid data may be, for example, (i) incomplete or insufficient for running the NDDTR model 120, (ii) data having significant outliers (e.g., values outside expected ranges; an intelligence quotient (IQ) of 180 could be indicative of an outlier); (iii) or combinations thereof. For example, an inconclusive output 131 may further indicate to a healthcare professional or other user that the patient data may have been input incorrectly into the NDDTR model 120, and thus the inputs should be double checked and corrected; and/or the patient data may fall on outlier values for certain ranges, and thus the patient should undergo further assessment in an attempt to clarify the outlier values. In some cases, the assessments that yielded outlier values may be repeated for validation.

Additionally or alternatively, in some embodiments, the NDDTR model 120 may be configured to validate data associated with the subject, for example, such that only valid data 116 are considered by the NDDTR model 120 and/or such that invalid data 117 are disregarded by the NDDTR model 120.

In various embodiments, for example, as illustrated in FIGS. 1 and 2, the data associated with the subject that is used as the input 110 to the NDDTR model 120 may comprise demographic data, schooling data, family medical data, prior therapy data, observational assessment data, questionnaire data, medication data, goals data, or combinations thereof. For example, in some embodiments, the data associated with the subject that are used as the input 110 to the NDDTR model 120 may comprise two or more of the demographic data, schooling data, family medical data, prior therapy data, observational assessment data, questionnaire data, medication data, goals data, or combinations thereof, additionally or alternatively, three or more of the demographic data, schooling data, family medical data, prior therapy data, observational assessment data, questionnaire data, medication data, goals data, or combinations thereof, additionally or alternatively, four or more of the demographic data, schooling data, family medical data, prior therapy data, observational assessment data, questionnaire data, medication data, goals data, or combinations thereof, additionally or alternatively, five or more of the demographic data, schooling data, family medical data, prior therapy data, observational assessment data, questionnaire data, medication data, goals data, or combinations thereof, additionally or alternatively, six or more of the demographic data, schooling data, family medical data, prior therapy data, observational assessment data, questionnaire data, medication data, goals data, or combinations thereof, or, additionally or alternatively, each of the additionally or alternatively, four or more of the demographic data, schooling data, family medical data, prior therapy data, observational assessment data, questionnaire data, medication data, goals data, or combinations thereof. In some embodiments, the inputs 110 may be obtained from applied behavior analysis (ABA) intake forms filled by the parents/guardians of the patients. The intake form contains a variety of questions that include information like demographics, behavioral assessment, skill assessment, medical history, or the like, of the subject.

Various examples of the various types of the data, each referred as a “data feature,” as may be associated with the subject are illustrated in Table 1:

TABLE 1

| | |
|---|---|
| Demographic | Age Data |
| Data | IQ Data |
| | Sex Data |
| | Handedness Data |
| | Race Data |
| | Ethnicity Data |
| | Socioeconomic Status Data |
| | Financial Data |
| | Monetary Income Data |
| | Monetary Savings Data |
| | Parental and/or Custodial Employment Data |
| | Parental and/or Custodial Education Data |
| | Health Insurance Data |
| | Health Insurance Provider Data |
| | Primary language |
| Schooling | Indication of Whether the Subject Attends School |
| Data | Indication of Whether the Subject has been Assigned a |
| | School Aide |
| | Indication of Whether the Subject is a Part of a Special |
| | Education Program |
| | School Grade Data |
| | Indication of Whether the Subject Receives any Special |
| | School Services |
| | Indication of Whether the Subject Receives Additional |
| | Services as part of a Special Education Program |
| Family | Indication as to the Presence or Absence of Depression or |
| Medical Data | Manic-Depression in an Immediate Family Member |
| | Indication as to the Presence or Absence of Substance |
| | Abuse or Dependence in an Immediate Family Member |
| | Indication as to the Presence or Absence of an Anxiety |
| | Disorder in an Immediate Family Member |
| | Indication as to the Presence or Absence of ADHD in an |
| | Immediate Family Member |
| | Indication as to the Presence or Absence of a Learning |
| | Disability in an Immediate Family Member |
| | Indication as to the Presence or Absence of Psychosis or |
| | Schizophrenia in an Immediate Family Member |
| Prior | Indication of the Subject Having Previously Received |
| Therapy Data | ABA Therapy |
| | Indication of the Subject Having Previously Received |
| | Occupational Therapy |
| | Indication of the Subject Having Previously Received |
| | Speech Therapy |
| | Indication of Type of ABA Therapy Previously Received |
| | by the Subject |
| | Indication of Duration of ABA Therapy Previously |
| | Received by the Subject |
| | Indication of Amount of ABA Therapy Previously |
| | Received by the Subject |
| | Indication of the Subject Having Previously Received |
| | Physical Therapy |
| | Indication of the Subject Having Previously Received any |
| | Therapy other than ABA Therapy, Speech Therapy, |
| | Occupational Therapy, or Physical Therapy |
| Observational | Indication of the Subject's Tendency Toward Aggressive |
| Assessment Data | Behavior |
| | Indication of the Frequency and/or Severity of the |
| | Subject's Aggressive Behavior |
| | Indication of the Subject's Tendency Toward Engaging in |
| | Self-Injury Behavior |
| | Indication of the Frequency and/or Severity of the |
| | Subject's Self-Injury Behavior |
| | Indication of the Subject's Tendency Toward Stereotypy |
| | Indication of the |
| | Frequency and/or Severity of the Subject's Stereotypy |
| | Indication of the Subject's Tendency Toward Destructive |
| | Behaviors |
| | Indication of the Frequency and/or Severity of the |
| | Subject's Destructive Behaviors |
| | Indication of the Frequency and/or Severity of the |
| | Subject's Destruction of Property |
| | Indication of the Consequences Implemented by a |
| | Caregiver of the Subject Responsive to Negative Behavior |
| | Indication of the Subject's Ability to be Understood |
| | Indication of the Subject's Ability to understand others |
| | Indication of Variety of Foods Eaten by the Subject |
| | Indication of the Subject's Ability use a Toilet |
| | Independently |
| | Indication of the Subject's Ability to Bathe Independently |
| | Indication of Stimulatory Behaviors Exhibited by the |
| | Subject |
| | Indication of Whether the Subject can be Described as |
| | Easy-Going or Going with the Flow |
| | Indication of Whether the Subject is Anxious or Easily |
| | Upset by Things that Would not Regularly Upset Others |
| | in Otherwise Similar Circumstances |
| | Indication of Whether the Subject Follows |
| | Simple Directions in a Home Setting |
| Medication | Indication of a Medication for Sleep Used by the Subject |
| Data | Indication of a medication for allergies Used by the |
| | Subject |
| | Indication of a Medication for ASD Used by the Subject |
| | Indication of a Medication for ADHD Used by the Subject |
| | Indication of a Medication for Anxiety Used by the |
| | Subject |
| | Indication of a Medication for Depression Used by the Subject |
| | Indication of a Medication for a Behavior or Mood-Related Condition |
| | Used by the Subject |
| Goals | Indication of a Goal of Improved Communication Skills |
| Data | Indication of a Goal of Improved Diet |
| | Indication of a Goal of Increased Independence |
| | Indication of a Goal of Improved Ability to Express |
| | Emotions |
| | Indication of a Goal of Improved Social Skills |
| | Indication of a Goal of Improved Ability to Participate in |
| | Family Activities |
| | Indication of a Goal of Decreased Challenging Behaviors |
| | Indication of a Goal of Getting Along Better with Parents |
| | and/or Siblings |
| | Indication of a Goal of Learning Toilet Training |
| | Indication of a Goal of Learning |
| | New Ways to Leave Non- |
| | Preferred Activities |
| | Indication of a Goal of Doing What They are Told Without |
| | Responding Inappropriately |
| | Indication of a Goal of Keeping Their Body and Others |
| | Around Them Safe |
| | Indication of a Goal of Increased Participation in General |
| | Education Classrooms or Settings |
| | Indication of a Goal of Increased Flexibility and/or Being Less Rigid |

For example, wherein the demographic data comprise age data, IQ data, sex data, handedness data, race data, ethnicity data, socioeconomic status data, financial data, monetary income data, monetary savings data, parental and/or custodial employment data, parental and/or custodial education data, health insurance data, health insurance provider data, or combinations thereof. Also for example, the schooling data comprise an indication of whether the subject attends school, an indication of whether the subject has been assigned a school aide, an indication of whether the subject is a part of a special education program, school grade data, an indication of whether the subject receives any special school services, an indication of whether the subject receives additional services as part of a special education program, or combinations thereof. Also for example, the family medical data comprise an indication as to the presence or absence of depression or manic-depression in an immediate family member, an indication as to the presence or absence of substance abuse or dependence in an immediate family member, an indication as to the presence or absence of an anxiety disorder in an immediate family member, an indication as to the presence or absence of ADHD in an immediate family member, an indication as to the presence or absence of a learning disability in an immediate family member, an indication as to the presence or absence of psychosis or schizophrenia in an immediate family member, or combinations thereof. Also for example, the prior therapy data comprise an indication of the subject having previously received ABA therapy; an indication of the subject having previously received occupational therapy; an indication of the subject having previously received speech therapy; an indication of type of ABA therapy previously received by the subject; an indication of duration of ABA therapy previously received by the subject; an indication of amount of ABA therapy previously received by the subject; an indication of the subject having previously received physical therapy; an indication of the subject having previously received any therapy other than ABA therapy, speech therapy, occupational therapy, or physical therapy; or combinations thereof. Also for example, the data associated with the subject comprise an indication of the subject's tendency toward aggressive behavior, an indication of the frequency and/or severity of the subject's aggressive behavior, an indication of the subject's tendency toward engaging in self-injury behavior, an indication of the frequency and/or severity of the subject's self-injury behavior, an indication of the subject's tendency toward stereotypy, an indication of the frequency and/or severity of the subject's stereotypy, an indication of the subject's tendency toward destructive behaviors, an indication of the frequency and/or severity of the subject's destructive behaviors, an indication of the frequency and/or severity of the subject's destruction of property, an indication of the consequences implemented by a caregiver of the subject responsive to negative behavior, an indication of the subject's ability to be understood, an indication of the subject's ability to understand others, an indication of variety of foods eaten by the subject, an indication of the subject's ability use a toilet independently, an indication of the subject's ability to bathe independently, an indication of stimulatory behaviors exhibited by the subject, an indication of whether the subject can be described as easy-going going with the flow, an indication of whether the subject is anxious or easily upset by things that would not regularly upset others in otherwise similar circumstances, an indication of whether the subject follows simple directions in a home setting, or combinations thereof. Also for example, the medication data comprise an indication of any medication used by the subject, wherein the medication data comprise an indication of a medication for sleep used by the subject, an indication of a medication for allergies used by the subject, an indication of a medication for ASD used by the subject, an indication of a medication for ADHD used by the subject, an indication of a medication for anxiety used by the subject, an indication of a medication for depression used by the subject, an indication of a medication for a behavior or mood related condition used by the subject, or combinations thereof. Also for example, the goals data comprise an indication of a goal of improved communication skills, an indication of a goal of improved diet, an indication of a goal of increased independence, an indication of a goal of improved ability to express emotions, an indication of a goal of improved social skills, an indication of a goal of improved ability to participate in family activities, an indication of a goal of decreased challenging behaviors, an indication of a goal of getting along better with parents and/or siblings, an indication of a goal of learning toilet training, an indication of a goal of learning new ways to leave non-preferred activities, an indication of a goal of doing what they are told without responding inappropriately, an indication of a goal of keeping their body and others around them safe, an indication of a goal of increased participation in general education classrooms or settings, an indication of a goal of increased flexibility and/or being less rigid, of combinations thereof.

In some embodiments, the data associated with the subject may be configured for input into a computing system, for example, such that the data associated with the subject may be evaluated via the NDDTR model 120. The data associated with the subject, also referred to as data features, may be represented and/or formatted in any suitable way. For example, the data associated with the subject comprise structured data, that is, data having a standardized format. Data feature processing can be performed prior to inputting the data into the NDDTR model 120. For example, some text data may be converted into binary (true vs. false, yes vs. no) data to display the presence or the absence of a certain feature for a particular subject. In some aspects, two or more input features may be combined prior to input into the NDDTR model. In other aspects, two or more input features may be combined subsequent to input into the NDDTR model. In yet other aspects, some input features may be combined prior to input into the NDDTR model; and other input features may be combined subsequent to input into the NDDTR model. In some embodiments, a data feature may be one-hot encoded, (for example, “Sex” represented as a combination of two new data features “Male” and “Female”) or converted into an ordinal type (for example, “How severe is the child's aggressive behavior?”: Mild=1, Moderate=2, Severe=3).

For example, the data from the ABA intake forms may include behavioral and/or skill assessments, often completed by a parent or caregiver, often including “Yes/No” and textual questions. In order for such data to be useful for the model, the data may be converted into categorical and binary inputs that could be converted into numerical vectors to be used as inputs to the machine learning model. In some embodiments, various data features may be aggregated into combined values or scores to represent information with regard to the subject while reducing the dimensionality of the data, that is, the number of data features. For instance, the various questions about social behaviors of a patient may be combined into a single score representing positive social behavior.

As an example of the ways in which various of the data associated with the subject may be configured for input, an “Aggression Score” (e.g., a single or individual data feature) may be derived from three distinct data features (variables) by multiplying their values, such as: “Does the child display aggression?” with possible values Yes (1) and No (0), “How frequently does the child exhibit aggression?” with possible values Less often than weekly (0), Weekly (1), Daily (2) and Hourly (3), and “How severe is the child's aggressive behavior?” with possible values of Mild (1), Moderate (2), Severe (3). An individual who exhibited moderate aggressive behavior on a daily basis would have a value of 4 that is, the product of 1×2×2, for the “Aggression Score.” If an individual does not exhibit any aggressive behavior or exhibits aggressive behavior less often than weekly, the Aggression Score is 0.

Additionally, data features exhibiting a high degree of correlation with one or more other features may be combined or eliminated, for example, where two or more features exhibit a correlation or correlation coefficient of at least about 75%, additionally or alternatively, at least about 80%, additionally or alternatively, at least about 85%, additionally or alternatively, at least about 90%, additionally or alternatively, at least about 95%. Not intending to be bound by theory, relatively highly correlated features may provide similar information to the model and thus, removing relatively highly correlated features may help reduce the dimensionality of the data, address concerns of computational complexity without hampering the model's performance, etc. If not mitigated, relatively high dimensionality can also lead to difficulties in the model's ability to identify the features of most importance.

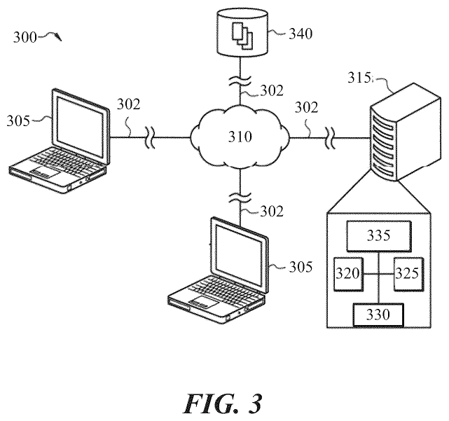

In some embodiments, the NDDTR model 120 may be characterized as a machine learning model. An example of the implementation of a machine learning model, for example, the NDDTR model as disclosed herein is illustrated in the context of FIG. 3. For example, FIG. 3 illustrates an embodiment of a computing system 300 that includes a number of clients 305, a server system 315, and a data repository 340 communicably coupled through a network 310 by one or more communication links 302 (e.g., wireless, wired, or a combination thereof). The computing system 300, generally, can execute applications and analyze data received from sensors, such as may be acquired in the performance of the methods disclosed herein. For instance, the computing system 300 may execute a machine learning model 335 as disclosed herein.

In general, the server system 315 can be any server that stores one or more hosted applications, such as, for example, the machine learning model 335. In some instances, the machine learning model 335 may be executed via requests and responses sent to users or clients within and communicably coupled to the illustrated computing system 300. In some instances, the server system 315 may store a plurality of various hosted applications, while in other instances, the server system 315 may be a dedicated server meant to store and execute only a single hosted application, such as the machine learning model 335.

In some instances, the server system 315 may comprise a web server, where the hosted applications represent one or more web-based applications accessed and executed via network 310 by the clients 305 of the system to perform the programmed tasks or operations of the hosted application. At a high level, the server system 315 can comprise an electronic computing device operable to receive, transmit, process, store, or manage data and information associated with the computing system 300. The server system 315 illustrated in FIG. 3 can be responsible for receiving application requests from one or more client applications associated with the clients 305 of the computing system 300 and responding to the received requests by processing the requests in the associated hosted application and sending the appropriate response from the hosted application back to the requesting client application.

In addition to requests from the clients 305, requests associated with the hosted applications may also be sent from internal users, external or third-party customers, other automated applications, as well as any other appropriate entities, individuals, systems, or computers. As used in the present disclosure and as described in more detail herein, the term “computer” is intended to encompass any suitable processing device, such as an electronic computing device. For example, although FIG. 3 illustrates a single server system 315, a computing system 300 can be implemented using two or more server systems 315, as well as computers other than servers, including a server pool. The server system 315 may be any computer or processing device such as, for example, a blade server, general-purpose personal computer (PC), Macintosh, workstation, UNIX-based workstation, or any other suitable device. In other words, the present disclosure contemplates computers other than general-purpose computers, as well as computers without conventional operating systems. Further, the illustrated server system 315 may be adapted to execute any operating system, including Linux, UNIX, Windows, MacOS, or any other suitable operating system. In some embodiments, the server system 315 comprises a cloud-based server, an edge server, or a combination thereof. For example, the electronic computing device may comprise an edge computing device, a cloud computing device, or both.

In the illustrated embodiment, and as shown in FIG. 3, the server system 315 includes a processor 320, an interface 330, a memory 325, and the machine learning model 335. The interface 330 is used by the server system 315 for communicating with other systems in a client-server or other distributed environment (including within computing system 300) connected to the network 310 (e.g., clients 305, as well as other systems communicably coupled to the network 310). Generally, the interface 330 comprises logic encoded in software and/or hardware in a suitable combination and operable to communicate with the network 310. More specifically, the interface 330 may comprise software supporting one or more communication protocols associated with communications such that the network 310 or interface's hardware is operable to communicate physical signals within and outside of the illustrated computing system 300.

Although illustrated as a single processor 320 in FIG. 3, two or more processors may be used according to particular needs, desires, or particular embodiments of computing system 300. Each processor 320 may be a central processing unit (CPU), a blade, an application-specific integrated circuit (ASIC), a field-programmable gate array (FPGA), or another suitable component. Generally, the processor 320 executes instructions and manipulates data to perform the operations of server system 315 and, specifically, the machine learning model 335. Specifically, the server's processor 320 executes the functionality required to receive and respond to requests from the clients 305 and their respective client applications, as well as the functionality required to perform the other operations of the machine learning model 335.

Regardless of the particular implementation, “software” may include computer-readable instructions, firmware, wired or programmed hardware, or any combination thereof on a tangible medium operable when executed to perform at least the processes and operations described herein. Each software component may be fully or partially written or described in any appropriate computer language including C, C++, C #, Java, Visual Basic, assembler, Perl, any suitable version of 4GL, Python, as well as others. It will be understood that while portions of the software implemented in the context of the embodiments disclosed herein may be shown as individual modules that implement the various features and functionality through various objects, methods, or other processes, the software may instead include a number of sub-modules, third-party services, components, libraries, and such, as appropriate. Conversely, the features and functionality of various components can be combined into single components as appropriate. In the illustrated computing system 300, processor 320 executes one or more hosted applications on the server system 315.

At a high level, the machine learning model 335 is any application, program, module, process, or other software that may execute, change, delete, generate, or otherwise manage information according to the present disclosure, particularly in response to and in connection with one or more requests received from the illustrated clients 305 and their associated client applications. In certain cases, only one machine learning model 335 may be located at a particular server system 315. In others, a plurality of related and/or unrelated modeling systems may be stored at a server system 315, or located across a plurality of other server systems 315, as well. In certain cases, computing system 300 may implement a composite hosted application. For example, portions of the composite application may be implemented as Enterprise Java Beans (EJBs) or design-time components may have the ability to generate run-time implementations into different platforms, such as J2EE (Java 2 Platform, Enterprise Edition), ABAP (Advanced Business Application Programming) objects, or Microsoft's .NET, among others. Additionally, the hosted applications may represent web-based applications accessed and executed by clients 305 or client applications via the network 310 (e.g., through the Internet).

Further, while illustrated as internal to server system 315, one or more processes associated with machine learning model 335 may be stored, referenced, or executed remotely. For example, a portion of the machine learning model 335 may be a web service associated with the application that is remotely located, while another portion of the machine learning model 335 may be an interface object or agent bundled for processing at a client 305 located remotely. Moreover, any or all of the machine learning model 335 may be a child or sub-module of another software module or enterprise application (not illustrated) without departing from the scope of this disclosure. Still further, portions of the machine learning model 335 may be executed by a user working directly at server system 315, as well as remotely at clients 305.

The server system 315 also includes memory 325. Memory 325 may include any memory or database module and may take the form of volatile or non-volatile memory. The illustrated computing system 300 of FIG. 3 also includes one or more clients 305. Each client 305 may be any computing device operable to connect to or communicate with at least the server system 315 and/or via the network 310 using a wired or wireless connection.

The illustrated data repository 340 may be any database or data store operable to store data, such as data of the type disclosed herein as associated with one or more subjects. Generally, the data may comprise inputs to the machine learning model 335, historical information, operational information such as features, and/or output data from the machine learning model 335.

The functionality of one or more of the components disclosed with respect to FIG. 3, such as the server system 315 or the clients 305, can be carried out on a computer or other device comprising a processor (e.g., a desktop computer, a laptop computer, a tablet, a server, a smartphone, smartwatch, or some combination thereof). Generally, such a computer or other computing device may include a processor (which may be referred to as a central processor unit or CPU) that is in communication with memory devices including secondary storage, read-only memory (ROM), random access memory (RAM), input/output (I/O) devices, and network connectivity devices. The processor may be implemented as one or more CPU chips.

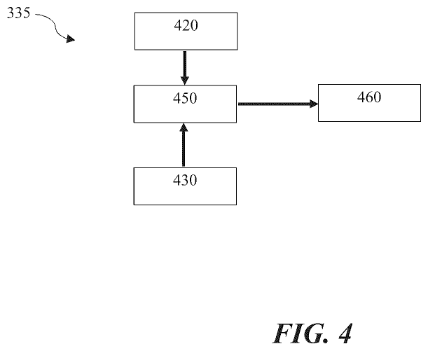

FIG. 4 depicts an example of the machine learning model 335 of FIG. 3. In the embodiment of FIG. 4, the machine learning model 335 comprises a machine learning module 450 coupled to one or more data stores, for example, data within the data repository 340. For instance, in the embodiment of FIG. 4, the data within the data repository 340 of FIG. 3 may include data from a training data store 420 and/or other inputs 430, as will be disclosed herein.

The machine learning module 450 can access data, such as data from the training data store 420, and receive inputs 430, and provide an output 460 based upon the inputs 430 and data retrieved from the training data store 420. Generally, the machine learning module 450 utilizes data stored in the training data store 420, for example, data of the type disclosed herein as data associated with a subject, to enable the resulting trained model (for example, the NDDTR model disclosed herein) to evaluate data associated with a subject, for example, to predictively determine a therapy recommendation comprising a standard of care. For example, the trained model may, in some embodiments, be characterized as a prediction algorithm.

Generally, the machine learning module 450 is a learning machine exhibiting “artificial intelligence” capabilities. For example, the machine learning module 450 may utilize algorithms to learn via inductive inference based on observing data that represents incomplete information about statistical phenomenon and generalizes it to rules and to make predictions on missing attributes or future data. Further, the machine learning module 450 may perform pattern recognition, in which the machine learning module 450 “learns” to automatically recognize complex patterns, to distinguish between exemplars based upon varying patterns, and to make intelligent predictions. In some embodiments, the machine learning module 450 can include or be accompanied by an optimization algorithm, like genetic algorithm (GA), ant colony optimization algorithm (ACO), simulated annealing (SA), etc. to increase the model accuracy and narrow down the data used to allow the machine learning module 450 to operate efficiently, even when large amounts of historical training data are present, and/or when complex input parameters are present.

The machine learning module 450 can comprise and/or implement any suitable machine learning algorithm or methodology, examples of which may include, but are not limited to, artificial neural networks (ANNs), deep neural networks (DNNs), deep reinforcement learning, convolutional neural networks (CNNs), a deep learning model, a generative adversarial network (GAN) model, a computational neural network model, a recurrent neural network (RNN) model, a perceptron model, decision trees such as a classical tree machine learning model, a decision tree type model, support vector machines, a regression type model, a classification model, a reinforcement learning model, Bayesian networks, optimization algorithms, and the like, or combinations thereof.

For example, in a particular embodiment, the machine learning module 450 utilizes gradient-boosted tree machine learning, for example, implemented in Python. Generally, a gradient-boosted tree aggregates results from various decision trees to output prediction scores. A dataset being evaluated may be split into successively smaller groups within each decision tree, for example, such that each tree branch divides a subject into one of two groups according to their covariate value and a predetermined threshold. The end of the decision tree is a set of leaf nodes, each of which represents a therapy recommendation for a patient. As the model is trained, successive trees are developed in order to improve the accuracy of the model. Successive iterations of trees utilize gradient descent of the prior trees in order to minimize the error of the new tree that is formed. In some embodiments, gradient-boosted tree machine learning implicitly handles any missing values, for example, various data associated with a subject that are not present. For instance, during the training phase, the model may “learn” the optimal branch directions for missing values.

At a high level, the machine learning module 450 may receive inputs 430 comprising parameters and hyperparameters (e.g., constraints) as to the training of the machine learning model, to perform learning with respect to the training data. Generally, a “hyperparameter” refers to a value (e.g., constraint) supplied to the model prior to final model training that dictate the properties of the model which is to be ultimately trained. Examples, in the context of a gradient-boosted tree, might comprise tree depth, number of decision trees, learning rate, scale positive weight, alpha regularization parameter, gamma regularization parameter, or combinations thereof. A “parameter” refers to a value learned during the training process that dictate the way in which a model interacts with the input data. Examples of parameters might include weights and biases of a neural network.

In some embodiments, the machine learning module 450 may “learn” or be trained by processing the training data, more particularly, the data from the training data store 420. As the machine learning module 450 processes the training data, the machine learning module 450 may form one or more probability-weighted associations between the various known inputs and the respective outcomes. As training progresses, the machine learning module 450 may adjust weighted associations between various inputs, for example, according to a learning rule, in order to decrease the error between the inputs and their respective outputs. As such, the machine learning module 450 may increasingly approach target output(s) until the error is acceptable.

In some embodiments, at least a portion of the data stored in the training data store 420 may be characterized as “training data” that is used to train the machine learning model 335. As will be appreciated by the ordinarily-skilled artisan upon viewing the instant disclosure, although the Figures illustrate an aspect in which the training data are stored in a single “store” (e.g., at least a portion of the training data store 420), additionally or alternatively, in some embodiments the training data may be stored in multiple stores in one or more locations.

Additionally, in some embodiments, the training data (e.g., at least a portion of the data stored in the training data store 420) may be subdivided into two or more subgroups, for example, a training data subset, one or more evaluation and/or testing data subsets, or combinations thereof. The training data may include a plurality of batches of data, each batch representing a data for each of a plurality of scenarios. Each batch of data may include data associated with each of a plurality of training subjects, particularly, including known inputs (e.g., demographic data, schooling data, family medical data, prior therapy data, observational assessment data, questionnaire data, medication data, goals data, or combinations thereof, as disclosed herein) associated with known outcome(s), for example, a therapy recommendation for each of the respective, plurality of training subjects.

Various combinations of data features may be used to train the model, that is, as the training data. In some embodiments, the training data comprises from about 10 to about 75 different data features, additionally or alternatively, from about 10 to about 50 different data features, additionally or alternatively, from about 12 to about 30 different data features, additionally or alternatively, from about 24 to about 30 different data features. In some embodiments, the set of data features employed may be selected so as to discriminate between patients having distinct therapy needs. For example, feature selection techniques such as correlation analysis, univariate feature analysis, feature selection based on feature importance (e.g., SHAP values), forward feature selection, backward feature elimination, recursive feature elimination, exhaustive feature selection, single feature model (area under the receiver operating characteristic curve (AUROC)), and combinations thereof.

For example, in an embodiment, with respect to a given feature group, a feature may be removed or retained based on the heuristics obtained from these various feature selection methods. For example, from a given group of features, the features that are among the most important features based on the feature selection methods may be kept and the features that are not among the most important features and/or also have a very low single feature model AUROC score may be removed from each group.

In various embodiments, the training data may include data associated with a plurality of subjects (e.g., training subjects), generally including data of the type disclosed herein as data associated with a subject, more particularly, demographic data, schooling data, family medical data, prior therapy data, observational assessment data, questionnaire data, medication data, goals data, or combinations thereof. Additionally, the training data may also include an indication of therapy received by or recommended for a particular subject. In some embodiments, the data employed as training data may be taken from a publicly available dataset. The data used may be anonymized (e.g., de-identified), for example, to ensure compliance with various regulations concerning patient information. In addition, the dataset can also contain professional (e.g., “official”) therapy recommendations. In some aspects, the efficacy of a therapy recommendation for a particular subject can be assessed by evaluating the progress of that particular subject with respect to their intended therapy goals vs. an expected rate of progress.

In some embodiments, the inputs 430 can comprise one or more constraints or limitations that may affect the way in which the machine learning module 450 is trained, an example of which includes the selection of one or more hyperparameters. In various embodiments, the inputs 430 can be provided as separate inputs, as a single input, or as a vector or matrix of input values. In some embodiments, the inputs 430 may be received, for example, from a user. Based on the inputs 430, the machine learning module 450 may use the data stored in the training data store 420 to develop the machine learning model 335, such as the NDDTR model 120 disclosed herein with respect to FIGS. 1 and 2.

As such, in some embodiments, based on processing the training data, for example, data from the training data store 420, the machine learning module 450 may yield a trained machine learning model 335 (e.g., the NDDTR model 120) that is configured to evaluate data associated with the subject to determine and provide a therapy recommendation.

In some embodiments, the NDDTR model may be configured to output a score, for example, between 0 and 1, indicative of the result. By default, a threshold of 0.5 is used to differentiate between positive and negative class, meaning that if the NDDTR model outputs a score greater than or equal to 0.5 for a subject, the subject belongs within the positive class, indicating, for example, that the subject requires comprehensive therapy; likewise, if the NDDTR model outputs a score less than 0.5, the subject belongs to the negative class, indicating that the subject requires focused therapy. However, this threshold value does not have to be set to 0.5. For instance, in various embodiments, a threshold value can be any suitable value between 0 and 1, for example a threshold value effective to yield a desired sensitivity. For example, the threshold value can be 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or any other suitable fractional value between 0 and 1 that is effective to yield a desired sensitivity. In some embodiments, a threshold value can be a value effective to yield a desired sensitivity; for example a sensitivity from about 0.7 to about 1.0, alternatively from about 0.71 to about 0.99, alternatively from about 0.75 to about 0.95, alternatively from about 0.75 to about 0.99, alternatively from about 0.8 to about 0.95, alternatively from about 0.85 to about 0.95, alternatively equal to or greater than about 0.7, alternatively equal to or greater than about 0.8, or alternatively equal to or greater than about 0.9, with respect to the therapy recommendation.

In an embodiment, for example, where the machine learning module 450 utilizes gradient-boosted tree machine learning, the trained machine learning model 335 may be characterized as having a depth of at least 2 and not more than 10, additionally or alternatively, a depth of at least 2 and not more than 7, additionally or alternatively, a depth of not more than 6, additionally or alternatively, a depth of not more than 5, additionally or alternatively, a depth of not more than 4, additionally or alternatively, a depth of not more than 3. Additionally or alternatively, the gradient-boosted tree model comprises a plurality of decision trees (e.g., estimators), for example, at least 50 decision trees, additionally or alternatively, at least 100 decision trees, additionally or alternatively, at least 150 decision trees, additionally or alternatively, at least 200 decision trees, additionally or alternatively, about 50 to about 600 decision trees, additionally or alternatively, about 50 to about 400 decision trees, additionally or alternatively, about 50 to about 200 decision trees, additionally or alternatively, about 50 to about 150 decision trees, additionally or alternatively, about 75 to about 125 decision trees. Additionally or alternatively, the gradient-boosted tree model may have a learning rate of less than or equal to about 0.4, additionally or alternatively, less than or equal to about 0.3. Additionally or alternatively, the gradient-boosted tree model may have a scale positive weight of from about 0.1 to about 10, additionally or alternatively, from about 1 to about 5. Additionally or alternatively, the gradient-boosted tree model may have an alpha regularization parameter of from about 0 to about 1. Additionally or alternatively, the gradient-boosted tree model may have a gamma regularization parameter of from about 0 to about 1. The number of decision trees determines the number of rounds of boosting, for example, the number of successive trees which are developed in creating the model. Higher values for the number of decision trees would increase the risk of model overfitting, thus detracting from the generalizability of the model, which may be a desired quality for the model. Scale positive weight is tuned to manage the class imbalance (e.g., positive class vs. negative class) in the dataset. The scale positive weight hyperparameter represents the ratio of the positive to negative class samples utilized to build each of the weak learners in the model, allowing the model to sufficiently and effectively learn from the data of the class with a lower prevalence in the dataset. Learning rate determines how quickly the model adapts to the data as the data may be fed in to create each successive tree. Regularization parameters alpha and gamma may be used to generalize the models as they become more complex in order to find effective models that are both accurate and as simple as possible.

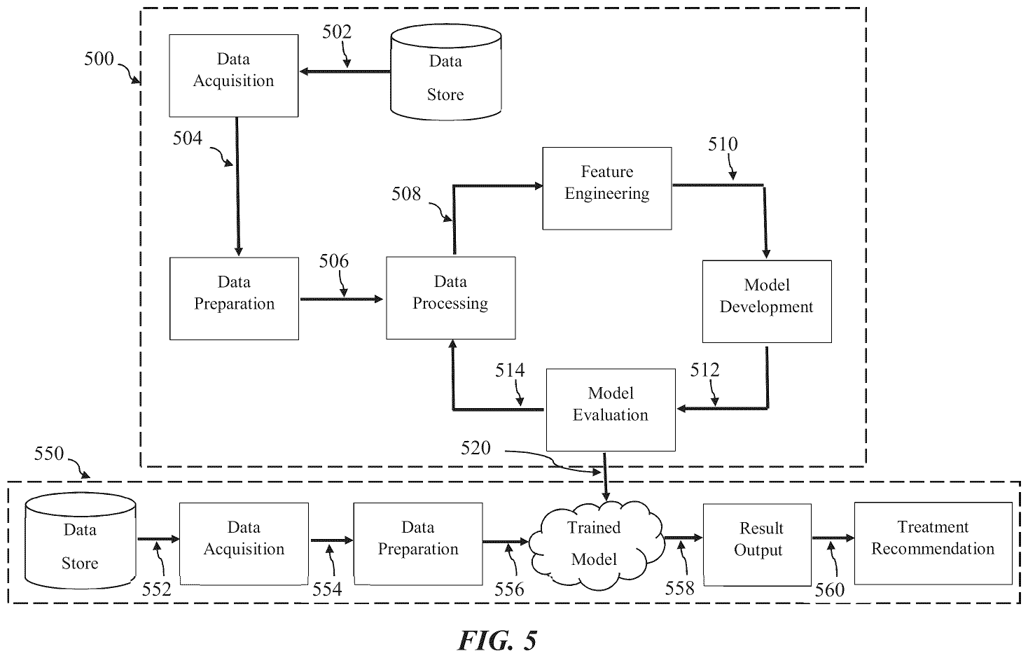

FIG. 5 illustrates methods related to the NDDTR model 120. Particularly, FIG. 5 illustrates both a method of training 500 the NDDTR model and a method of using the NDDTR model 550, for instance, in the determination and provision of a therapy recommendation.

Referring to FIG. 5, during the training 500, training data (e.g., a dataset) is acquired from the database or data store (step 502). As disclosed herein, the training data may include data taken or derived from ABA intake forms including demographic data, schooling data, family medical data, prior therapy data, observational assessment data, questionnaire data, medication data, goals data, or combinations thereof. The dataset may undergo exploratory data analysis, for example, which may be effective to evaluate the structure, distribution, and/or quality of the dataset (step 504). Additionally, the dataset may be processed, for example, filtered, to ensure that patient data that is severely out of distribution or has significant missingness which may skew the results is omitted. Additionally, for example, following this initial filtration, the training data may undergo further processing, including imputing or removing outliers and/or unidentified characters, and removing features that may be relatively highly correlated with other features, have a relatively high degree of missing values, or are not important (step 506). Additional features may also be extracted based upon examination of feature importance during preliminary model development, through advice/consultation on clinical judgment from experts in the field, or through some combination thereof. For example, textual data may be converted into binary features or categorical features may be broken into multiple binary features (step 508). The dataset may then be randomly divided into a training subset and a testing subset, such as a hold-out test set. For example, a hold-out test set may be formed by including a random percentage of the individuals from the total dataset (e.g., from about 15% to about 25%, alternatively from about 16% to about 24%, alternatively from about 17% to about 23%, alternatively from about 18% to about 22%, alternatively from about 19% to about 21%, or alternatively about 20% of the individuals from the total dataset). The testing subset may be maintained completely independent of the training process such that it is solely used to evaluate the performance of the resultant model so as to determine the model's efficacy.

In some embodiments, prior to training the model, one or more hyperparameters may be selected and/or optimized, for example, to determine the best (e.g. the most effective) hyperparameters for training utilizing a gradient-boosted tree learning model. As discussed herein, hyperparameters are elements of the machine learning model that dictate the training process and the specific way in which a machine learning model learns. As an example, in gradient-boosted tree learning, the depth of each tree (meaning how many features are evaluated to classify a data input) is a hyperparameter of the model. Different tree depths would alter the way in which model training occurs so a hyperparameter optimization might evaluate depths of 2, 3, 4, 5, 6, 7, or more to identify which one(s) leads to optimal model performance. The hyperparameter optimization may also include a multiple-fold cross-validation, for example, in order to evaluate the hyperparameter's performance on unseen data. The cross-validation may include 2, 3, 4, 5, 6, 7, or more folds. Following hyperparameter optimization, the model may be trained, for example, using the optimized hyperparameters (step 510). In order to evaluate the model's performance, the hold-out test subset may be passed through the model and the results may be analyzed. In some embodiments, the hold-out data subset may be exclusively used to evaluate the performance results, for example, in order to prevent any data leakage from the training data on the model's performance (step 512). Based on the results that we get from this evaluation, the steps associated with data preparation and processing, feature engineering, model training, and evaluation may be repeated (step 514) iteratively until satisfactory performance is demonstrated (steps 508, 510, 512, and 514), for example, as demonstrated by a desired sensitivity and/or specificity.

When the trained model demonstrates satisfactory performance, the model may then be deployed, for example, via a cloud server, at which time the model is able to be used during prospective settings (step 520). During the prospective setting, data associated with a subject to be evaluated may be acquired, for example, from a database, for example, which may include data obtained from ABA intake forms, and then cleaned and processed as similarly done with respect to the training data (steps 552 and 554). The data associated with the subject being evaluated may then be input into the machine learning model (for example, an NDDTR model served via a cloud device) to output a therapy recommendation, for example, a result including a standard of care (steps 556 and 558). The therapy recommendation (e.g., the result) may be displayed to the end user, which usually is the person evaluating the subject (step 560), for example, a healthcare provider. The prediction may be presented to a user (e.g., a healthcare professional) via a user interface. For example, the therapy recommendation may be presented graphically, in text, and/or as audio. In various embodiments, the user interface may include a graphical user interface (e.g., a screen and/or touch-screen), a speaker, or the like. For example, the user interface may be delivered via a user device (e.g., a desktop computer, a laptop computer, a tablet, a server, a smartphone, a smartwatch, or some combination thereof).

Additionally, in some embodiments, a method of using the NDDTR model may further include providing treatment to the subject receiving the therapy recommendation based upon the therapy recommendation. In various embodiments, the treatment provided to the subject can comprise ABA therapy, speech therapy, physical therapy, and the like, or combinations thereof.

The NDDTR model as disclosed herein may be advantageously employed in the determination and provision of a therapy recommendation to a subject having an NDD, for example, a subject having ASD. For instance, the NDDTR model demonstrates the unique potential to improve the process by which therapy recommendations are provided to a subject across all age groups.

Additionally, the disclosed NDDTR model allows for accurate determination and provision of a therapy recommendation for a subject, which conventionally involves a time-consuming and resource-intensive process, that can be achieved in a matter of minutes. Moreover, the disclosed NDDTR model empowers caregivers to provide a therapy recommendation much earlier than previously possible and implement the therapy recommendation as early as possible, which is highly desirable in the treatment of NDDs, particularly, in the treatment of ASD disorders such as autistic disorder, Asperger's disorder, PDD-NOS, etc. In an aspect, the NDDTR model as disclosed herein can provide for achieving a standardized therapy recommendation process for individuals having an NDD. In some aspects, the NDDTR model as disclosed herein can provide for achieving a standardized therapy recommendation process for individuals having ASD, for example regarding an ABA therapy recommendation of focused ABA therapy or comprehensive ABA therapy.

### EXAMPLES

The presently disclosed subject matter having been generally described, the following examples are given as particular aspects of the subject matter and to demonstrate the practice and advantages thereof. It is understood that the examples are given by way of illustration and are not intended to limit the specification or the claims in any manner.

### Example 1

An example of the model disclosed herein, particularly, the NDDTR model was trained with a dataset including phenotypic data including clinical, demographic, and assessment data for over 350 individuals, with approximately one third being individuals assigned to a comprehensive ABA therapy plan. Data were collected from parents/caregivers of individuals with an NDD, particularly, ASD, prior to the start of ABA therapy with the specific provider. The data contained a parent's assessment of the demographic information, child's behaviors, and social abilities at the time of enrolling in an ABA program. All patients had been diagnosed with ASD by a qualified healthcare provider (e.g., clinician). Filtering was performed to ensure data availability amongst the patients. The final dataset was filtered down to 359 individuals, which were randomly divided in a training set and a hold-out test set. Individuals of ages ranging from 1 to 50 years were included in the dataset. The hold-out test set was acquired by selecting a random 20% of the individuals from the total dataset. The hold-out test set remained completely independent of the training process and was solely used to evaluate model results to determine the algorithm's efficacy.

The following steps and techniques were used to prepare the data for training and testing the NDDTR algorithm.

The data were subjected to cleaning and conversion. Particularly, in order to clean the data obtained from the ABA intake forms, the following measures were taken:
a. All features and samples that were redundant or highly missing were removed.
b. All outlier data with values severely outside the distribution were removed.
c. If a pair of features had very high correlation, one of them was removed.
d. All textual fields were either converted to binary or categorical features.
e. Some features were combined to form a single feature while maintaining the information from the features that were combined.

The data were subjected to feature selection and processing. Particularly, in order to find the optimal set of features that would be able to discriminate between patients needing comprehensive and focused therapy plans, various feature selection techniques were used. The feature selection techniques used were correlation analysis, univariate feature analysis, based on feature importance, forward feature selection, backward feature elimination, recursive feature elimination, and exhaustive feature selection. The feature processing steps are discussed in the following section.

The steps used for feature processing are discussed below, for example, so as to reduce the dimensionality of the feature set and improve the performance of the model.
1. Conversion of the inputs in the form to numeric (categorical, binary) columns.
2. Removal of features with a high rate of missingness. Any features with a missingness of 50% or more were removed from the list of inputs.
3. Removal of a feature from pairs of features with a high magnitude of correlation (>0.85).
4. Combining binary features into a single numeric/ordinal feature.
   Example: Inputs relating to the assessment of social skills were converted into a single numeric value representing the level social skills of the individual.
5. Grouping of features based on various aspects of the patient's information in order to analyze and find the most important features in each group. The features were grouped into the following groups:
   Demographics
   Schooling Information
   Parents' Medical History
   Therapy
   Behavioral Assessment
   Consequences for Misbehavior
   Communication Skills
   Feeding and Drinking Habits
   Sleep and Wake Patterns
   Toileting and Bathing Skills
   Stimulatory/Restricted and Repetitive Behaviors
   Social Skills
   Expected Parent Goals
6. Collection of heuristic on predictive impact of features using various feature selection techniques. The various feature selection techniques used in this step were:
   Forward Feature Selection
   Backward Feature Elimination
   Exhaustive Feature Selection
   Feature Selection Based on feature importance (SHAP Values)
   Single Feature Model (AUROC)
      From each of the feature groups, data features were removed or retained based on the heuristics obtained from these various feature selection methods. From every group of features, the features that were among the most important features based on the feature selection methods were kept and the features that did not show up among the most important features and also had a very low single feature model AUROC score were removed from each group.
7. Final feature selection on the remaining set of features based on AUROC using an elimination method whereby, for example, running a model with a particular feature and the model performance is evaluated, such as via AUROC. The feature may be replaced and another feature is removed and the performance of the model is again evaluated. Finally, the feature which when removed improved the model performance or did not reduce the model performance is eliminated. The steps are repeated again for the remaining features. This method of recursive feature elimination is repeated until a high model performance is achieved with a set of features that cover the various aspects of the individual's data while omitting unnecessary or noisy features.
8. The final set of features is then used to build the final model.

For example, the AUROC-based feature selection started by building a baseline model using all the features remaining at before this step (before step 7 in the feature selection step). Models were then built by iteratively removing one feature from the feature set one at a time with replacement. The model used for this stage of feature selection used a XGBoost model with a fixed set of hyperparameters (a max depth of 2 with 100 decision trees (“estimators”) was used to ensure the model did not overfit). After going through each of the features, removing one at a time with replacement, the feature which yielded the largest AUROC value when removed was eliminated from the list of features, in order to avoid data overfitting (which would be detrimental to model performance and generalizability). This process was then repeated with the set of remaining features to identify and discard noisy, low predictive features. All the combination of features and the corresponding AUROC values were noted. After multiple iterations, the best combination of features that achieved the highest AUROC while capturing the various aspects of the individual's data was chosen as the final set of features to train the model.

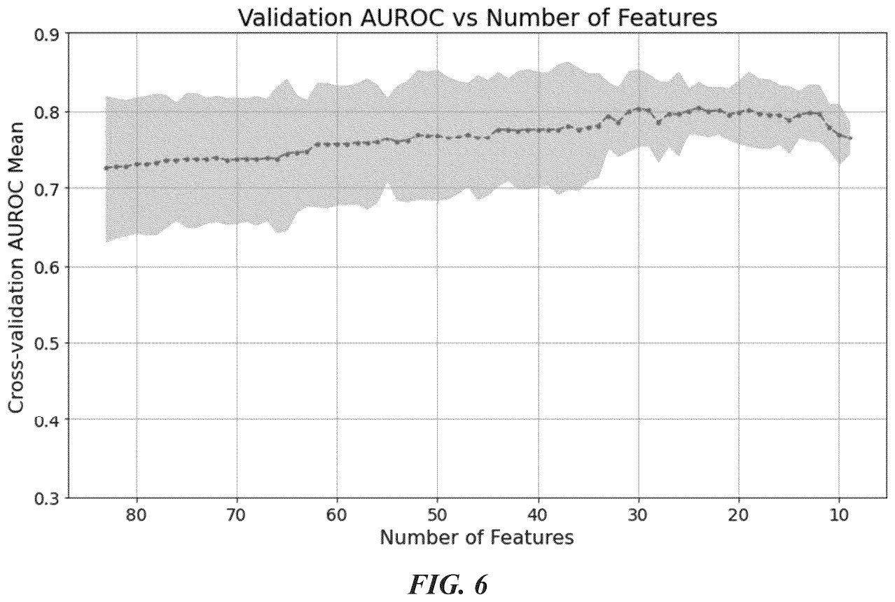

The results from the AUROC-based feature elimination method are shown in FIG. 6. From the initial set of over 80 features, as features are eliminated one after another based on the AUROC based elimination method disclosed herein, a gradual increase in AUROC was observed with a maximal value being achieved with 24 feature inputs. The average cross-validation AUROC approaching the maximal value attained during the process (˜0.80) occurred for three sets of features, particularly, feature sets with 30, 27 and 24 data features. In order to allow the model to operate based on as much information as possible, the set of 30 features was used as the final set of inputs.

Inputs for the NDDTR model were obtained from the ABA intake forms filled by the parents/guardians of the patients. The intake form contains a variety of questions that include information like demographics, behavioral assessment, skill assessment, medical history, and the like associated with the patient with ASD. Alternative versions of NDDTR model can be trained using any combination of these inputs. For instance, approximately 180 distinct data features, across various categories of data as disclosed herein, may be obtained from ABA intake forms.

Using the feature selection and processing as disclosed herein, the following combination of features were chosen for the NDDTR model:
Demographic and Schooling Information:
   Age
   Attends School?
   Grade (If attends school)
   Does the individual have a School Aide?
   Individual received additional services as part of an Individualized Education Plan (IEP)/Admission, Review, Dismissal (ARD) process
Parental Medical History:
   Mother or Father has history/presence of depression or manic-depression
   Mother or Father has history/presence of substance abuse or dependence
   Mother or Father has history/presence of anxiety disorders (OCD, phobias, etc.)
   Mother or Father has history/presence of ADHD
Therapy:
   Amount of prior ABA therapy (hours of therapy per week)
   Amount of prior ABA therapy (years)
   History of Occupational Therapy: Has the individual ever received Occupational Therapy?
   History of Speech Therapy: Has the individual ever received Speech Therapy?
Behavioral Assessment:
   Aggression Score: Level of the individual's engagement in aggressive behavior derived from the frequency and severity of aggression
   Does the individual engage in stereotypy?
   Stereotypy Score: Level of the individual's engagement in Stereotypical repetitive behavior derived from the frequency and severity of stereotypy behavior
   Destroy Property: Does the individual destroy property?
   Count of Consequences: How many types of consequences for misbehavior do the parents/caregivers use for the individual?
   Individual Understanding Others: Does the individual appear to understand his/her parents/caregiver?
   Stranger Understanding Individual: Can strangers usually understand the individual?
   Food Choices: Level of pickiness of individual regarding eating
   Toileting Independence: Individual's level of toileting ability
   Bathing Ability: Ability of individuals to bathe themselves
   Stimulatory Behavior/Restricted and Repetitive Behavior Count: How many types of stimulatory behaviors does the individual exhibit?
Expected Parent/Caregiver Goals: Goals which parents/caregivers hope to achieve through ABA therapy
   Improve communication skills
   Learn to eat healthier/more balanced diet
   Learn to be more independent
   Learn new ways to express frustration or when upset
   Learn new ways to leave non-preferred activities
Medical History
   Medication for Sleep
   Medication for Allergies

Using the final set of features outlined above, a XGBoost model was trained with the 288 data points in the training data set (which represent the information from about 80% of the subjects in the full dataset containing 359 subjects). XGBoost is a gradient-boosted tree ensemble method of machine learning which combines the estimates of simpler, weaker models—in this case, relatively shallow decision trees—to make predictions for a target. One of the benefits of using XGBoost is that it can implicitly handle missingness in the data. The model development is shown in FIG. 5 and included a 5-fold cross-validation on the training data set. A cross-validation method is a resampling method that uses different portions of the data to test and validate a model on different iterations. In this case, the 288 data points in the training set were divided into five equally-sized groups, after which a model was trained using four of these groups and validated using the fifth, remaining group. This was repeated using each of the five groups as a validation set. The method of cross-validation allows for building a model more robust to variability in the data.

Hyperparameters were optimized using a grid search method. The three main hyperparameters were tuned using the grid search method and included maximum tree depth, number of estimators, and scale positive weight. The maximum tree depth hyperparameter determines the complexity of the weak learners; that is, the tree depth hyperparameter limits the depth of the contributing decision trees. Lower range of values between 2 and 4 were selected for tuning maximum tree depth in order to develop a more conservative model. The number of decision trees or estimators determines the number of rounds of boosting, that is, a method of combining the estimates of the weak learners by taking each weak learner sequentially and modeling based on the error of the preceding weak learner. A relatively higher value for the number of estimators would increase the risk of model overfitting. Thus, the search grid for the number of estimators was kept under 500. Scale positive weight is tuned to manage the class imbalance in the dataset. This hyperparameter's search grid was set with values close to the ratio of the counts of two classes. The values for maximum tree depth, number of estimators, and scale positive weight after hyperparameter tuning were set to 2, 100, and 2.6, respectively. Once the hyperparameters were tuned, a final model was trained using all the 288 data points in the training dataset. The final model was evaluated on the testing set.

The NDDTR model performance in determining/predicting the ABA therapy treatment indicates a strong ability to distinguish between the need for various therapies (e.g., between a focused ABA treatment plan and a comprehensive ABA treatment plan). As the conventional ABA treatment plan determination process is multifactorial and encompasses a relatively high level of clinical judgment, no direct comparator exists to measure the NDDTR model against. Consequently, a comparator was developed, wherein the comparator encompassed the features that are specified by the Behavior Analyst Certification Board (BACB) guidelines to contribute to the decision of a focused vs. a comprehensive ABA care plan (e.g., ABA therapy plan, ABA treatment plan, ABA therapy treatment plan, etc.).

The features selected for the comparator encompassed (per BACB guidelines) the types of behaviors exhibited by an individual, the number of behaviors exhibited by the individual, and the number of targets to be addressed for that particular individual. The comparator accounted for the following features as inputs into the comparator: age, restricted and repetitive behaviors, social and communication behaviors, listening skills, aggressive behaviors, and total number of goals to be addressed. These features were utilized in combination by the comparator to determine which care plan should be recommended, as follows. To combine the inputs to the comparator in order to generate a determination of a focused vs. a comprehensive care plan, a linear regression function was constructed. This linear regression function took all of the inputs and generated an output score, which was a linear combination of the inputs. A linear regression function was used as a proxy for the manual assessment process that the BCBA follows (based on the features outlined in the BACB guidelines) in order to determine whether an individual should receive focused ABA therapy or comprehensive ABA therapy. It should be noted that the calculations and data analysis done by the machine learning model that is used by the NDDTR model are far too complex to be performed manually by any individual. Scores generated by the linear regression function were then compiled and a cutoff was selected to determine which scores of the comparator indicated a focused care plan and which scores indicated a comprehensive care plan. This cutoff was selected to provide a balance of the performance of the linear regression function in differentiating between focused vs. comprehensive therapy plans.

AUROC was used as a performance metric to evaluate the NDDTR model. AUROC is a performance metric of discrimination, that is, it conveys the NDDTR model's ability to discriminate between classes (patients requiring a comprehensive therapy plan as compared to patients requiring a focused therapy plan). An AUROC greater than 0.5 means that the model will correctly assign a relatively higher absolute risk to a randomly selected patient with an event (patient requiring a comprehensive therapy plan) than to a randomly selected patient without an event (patient requiring a focused therapy plan). An AUROC of 0.50 is equivalent to random coin flip or no discrimination. The NDDTR model achieved an AUROC of 0.895.

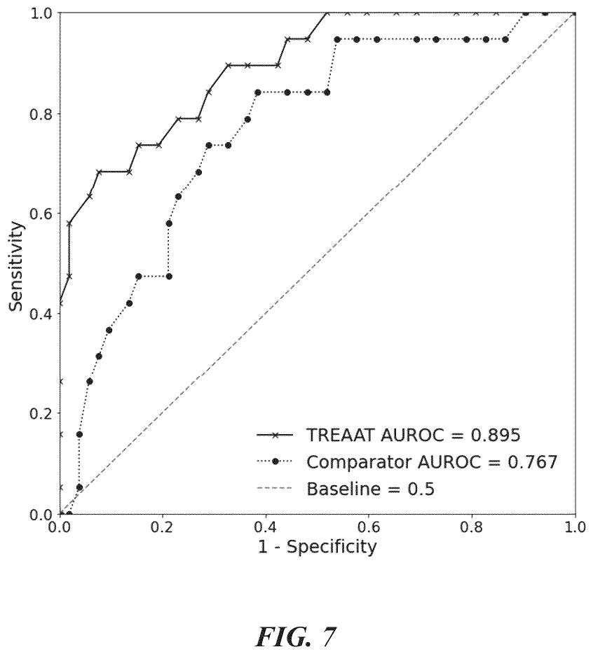

FIG. 7 shows the receiver operator characteristic (ROC) curve for the operation of the NDDTR model. The ROC curve is constructed by plotting “True Positive Rate (TPR)” or Sensitivity on the y-axis and “False Positive Rate (FPR)” or (1—Specificity) on the x-axis at different threshold values. A threshold value is a value that is used to separate the positive class and negative class. The NDDTR model outputs a score between 0 and 1. Therefore, there are infinite threshold values that can be used to differentiate between two classes. For example, if the threshold value is 0.5, patients with model output values greater than and equal to 0.5 are classified as requiring a comprehensive therapy plan, and values less than 0.5 are classified as requiring a focused therapy plan. At varying thresholds, the TPR and FPR of the model vary. Based on the expected TPR and FPR for the model, a threshold that achieves the desirable TPR and FPR can be chosen as the threshold. Plotting TPR and FPR at different thresholds yields the ROC curve. Selecting a threshold can be achieved by moving along the ROC curve. AUROC is the area under this 2-dimensional ROC curve. The NDDTR model achieved an AUROC of 0.895, making it a strong discriminator of the two classes. The complete list of performance metrics for NDDTR and the score representing the comparator is shown in Table 2 below. The NDDTR achieved a strong performance for classifying individuals as requiring comprehensive therapy or requiring focused therapy with an AUROC of 0.895 in the hold-out test set (confidence interval (CI): 0.811-0.962). The NDDTR model substantially outperformed the comparator which had an AUROC of 0.767 in the hold-out set (CI. 0.629-0.891).

In the context of the NDDTR model, the evaluation metrics are defined as follows. Sensitivity refers to the proportion of patients who the model deemed to require a comprehensive therapy plan among all those who actually received a comprehensive therapy plan. Specificity refers to the proportion of people who the model deemed to require a focused therapy plan among all those who actually received a comprehensive therapy plan. Positive Predictive Value (PPV) refers to the probability that following the model's outcome for the patient as requiring a comprehensive therapy plan, the patient actually received a comprehensive therapy plan (ground truth). Negative predictive value (NPV) refers to the probability that following the model's outcome for the patient as requiring a focused therapy plan, the patient actually received a focused therapy plan (ground truth). These metrics can be calculated as follows.

$$Sensitivity = \frac{\begin{matrix}No . of patients correctly classified by \\ the model as needing \\ comprehensive treament plan\end{matrix}}{\begin{matrix}No . of patients who received comprehensive \\ treatment plan ( ground truth )\end{matrix}} \\ Specificity = \frac{\begin{matrix}No . of patients correctly classified by \\ the model as needing \\ focused treament plan\end{matrix}}{\begin{matrix}No . of patients who received focused \\ treatment plan ( ground truth )\end{matrix}} \\ PPV = \frac{\begin{matrix}No . of patients correctly classified by \\ the model as needing \\ comprehensive treament plan\end{matrix}}{\begin{matrix}No . of patients who were classified by \\ the models as needing \\ comprehensive treament plan\end{matrix}} \\ NPV = \frac{\begin{matrix}No . of patients correctly classified by \\ the model as needing \\ focused treament plan\end{matrix}}{\begin{matrix}No . of patients who were classified by \\ the model as needing \\ focused treament plan\end{matrix}}$$

TABLE 2

| Performance Metrics | NDDTR | Comparator |
|---|---|---|
| AUROC (95% CI) | 0.895 (0.811-0.962) | 0.767 (0.629-0.891) |
| Sensitivity (95% CI) | 0.789 (0.673-0.906) | 0.789 (0.700-0.878) |
| Specificity (95% CI) | 0.808 (0.740-0.876) | 0.635 (0.571-0.698) |
| PPV (95% CI) | 0.600 (0.478-0.722) | 0.441 (0.360-0.522) |
| NPV (95% CI) | 0.913 (0.861-0.965) | 0.892 (0.843-0.940) |

Notably, conventional definitions for binary classification metrics designate positive and negative classes as the presence or absence, respectively, of a condition or other label. In contrast to the conventional definitions, in Table 2, a ground truth indication of comprehensive treatment need is taken as to the “positive class,” and a ground truth indication of focused treatment need is equivalent to the “negative class,” as described in more detail below.

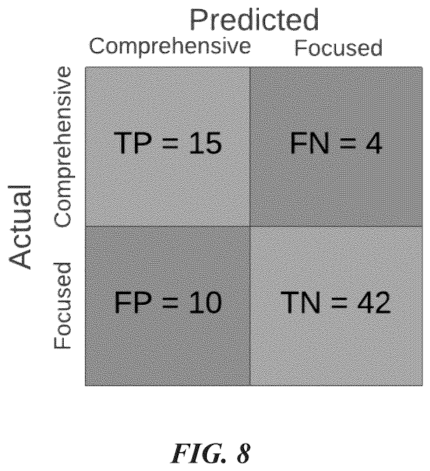

A confusion matrix that summarizes the prediction results of the NDDTR model on classifying patients requiring a comprehensive therapy plan as opposed to a focused care plan is shown in FIG. 8. The number of correct and incorrect predictions are presented with count values and broken down by each class. The labels on the x-axis represent the outcomes of the model, and on the y-axis are the actual labels. The confusion matrix provides insight into the errors being made by the utilized classifier, as well as the types of errors that are being made. The top left and bottom right boxes represent the counts of patients that the model classified correctly, whereas the top right and bottom left boxes represent the counts of patients that the model misclassified.

For the same testing data and the same classifier, one can generate multiple confusion matrices at different operating points, for example, at a threshold that achieves a certain sensitivity and specificity. An operating point was selected to prioritize true positives (TPs) and limit false negatives (FNs). For instance, while a false positive (FP) result may provide comprehensive therapy to a patient that could significantly improve with just focused therapy, an FN may lead to insufficient therapy for an individual in need of comprehensive therapy, which could impactfully diminish the progress that particular individual could make. FIG. 8 shows the confusion matrix at the chosen operating point for which the metrics are reported in Table 2 (Sensitivity: 78.9%, Specificity: 80.8%). At this operating threshold, NDDTR classifies patients between the two classes with only 14 misclassifications out of the 71 total patients in the testing dataset. It should be noted that the majority of misclassifications are FPs (10 misclassifications, accounting for 71% of total misclassifications) and indicate comprehensive therapy for individuals that may only need focused therapy. As noted, an FP result may provide more therapy to an individual, which would still benefit the subject. A very small portion of the misclassifications are FNs (4 misclassifications, accounting for 29% of total misclassifications) and indicate focused therapy for individuals that may benefit from comprehensive therapy. TN=true negative in FIG. 8.

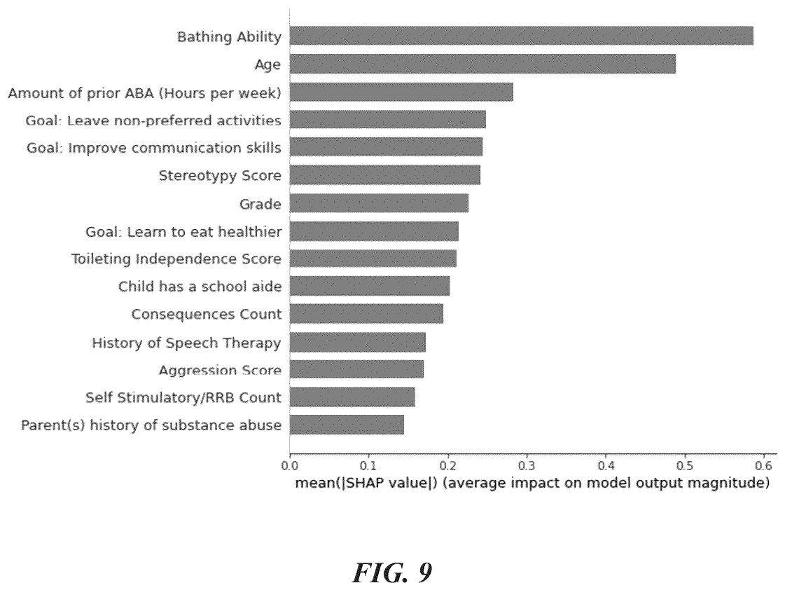

Feature Importance techniques rank features based on the effect they have on the model's outcomes. These techniques provide a score which implies the “importance” of each of the features where a higher score for a feature represents a larger effect of that feature on the model outcome. The feature importance of the features utilized by the NDDTR model was evaluated using SHAPley (SHapley Additive exPlanation, or “SHAP”) value plots as shown in the FIG. 9.

The SHAP summary plot of FIG. 9 displays the fifteen most-important features for the NDDTR model, with the features in the descending order of importance from top to bottom. The x-axis in the figure is the mean SHAP value, which indicates the average impact of the feature on model output. This value is the average marginal contribution of a feature value across all the possible combinations of features. For instance, FIG. 9 shows that the patient's bathing ability, age, and the amount of past ABA therapy (hours per week) are among the top 3 most important features that contribute to the NDDTR model's predictions.

Table 3 displays performance metrics demonstrating the discriminative capabilities of the NDDTR model by comparison with the comparator in three different age groups (i.e., 5 years, 5 to <8 years, >=8 years). Metrics used include AUROC, sensitivity, specificity, PPV, and NPV for the three age groups, demonstrating the superior performance of the NDDTR model in each of the three age groups. All metrics include a 95% CI.

TABLE 3

| | Performance Metrics | | | | | |
|---|---|---|---|---|---|---|
| | Age < 5 years | | Age 5 to < 8 years | | Age >= 8 years | |
| | Number of Patients | | | | | |
| | 25 | | 19 | | 27 | |
| | Model | | | | | |
| | NDDTR | Comparator | NDDTR | Comparator | NDDTR | Comparator |
| AUROC | 0.853 | 0.711 | 0.798 | 0.596 | 0.889 | 0.833 |
| (95% CI) | (0.698- | (0.500- | (0.550- | (0.288- | (0.615- | (0.609- |
| | 0.971) | 0.88) | 1.00) | 0.869) | 1.00) | 1.00) |
| Sensitivity | 0.857 | 0.857 | 0.500 | 0.500 | 0.667 | 0.667 |
| (95% CI) | (0.674- | (0.674- | (0.100- | (0.100- | (0.133- | (0.133- |
| | 1.00) | 1.00) | 0.900) | 0.900) | 1.00) | 1.00) |
| Specificity | 0.474 | 0.316 | 0.947 | 0.632 | 1.00 | 0.833 |
| (95% CI) | (0.249- | (0.107- | (0.847- | (0.415- | (1.00- | (0.684- |
| | 0.698) | 0.525) | 1.00) | 0.848) | 1.00) | 0.982) |
| PPV | 0.545 | 0.480 | 0.750 | 0.300 | 1.00 | 0.333 |
| (95% CI) | (0.337- | (0.284- | (0.326- | (0.016- | (1.00- | (0.044- |
| | 0.754) | 0.676) | 1.00) | 0.584) | 1.00) | 0.711) |
| NPV | 0.818 | 0.750 | 0.857 | 0.800 | 0.96 | 0.952 |
| (95% CI) | (0.590- | (0.450- | (0.707- | (0.598- | (0.883- | (0.861- |
| | 1.00) | 1.00) | 1.00) | 1.00) | 1.00) | 1.00) |

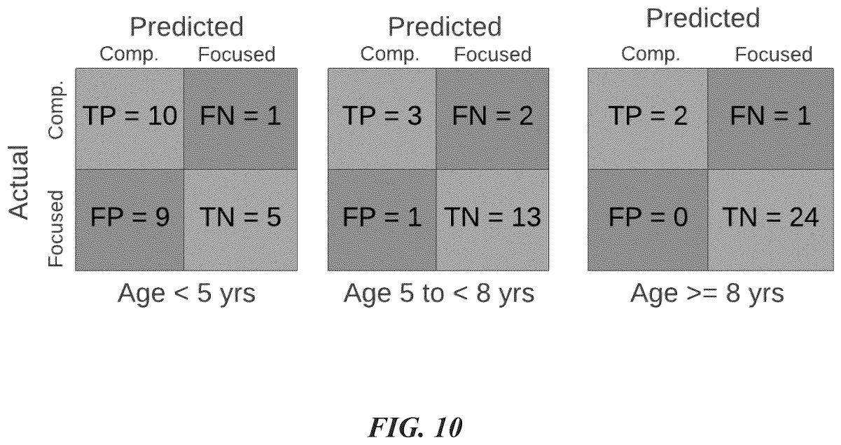

FIG. 10 displays confusion matrices providing a visual representation of the NDDTR model's output for the hold-out test dataset in three different age groups (i.e., <5 years, 5 to <8 years, ≥8 years). Comp.=comprehensive in FIG. 10.

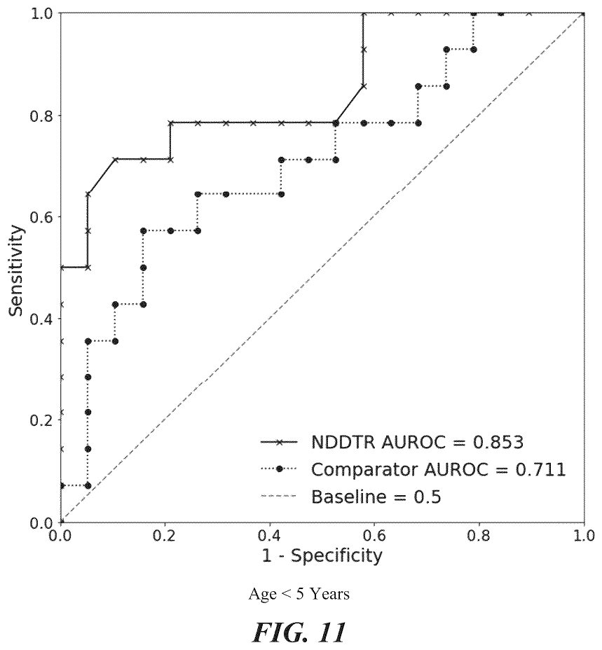

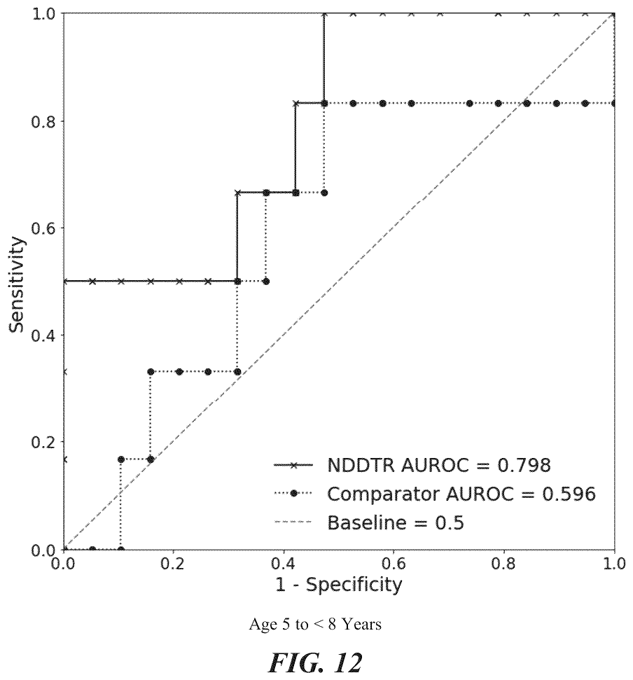

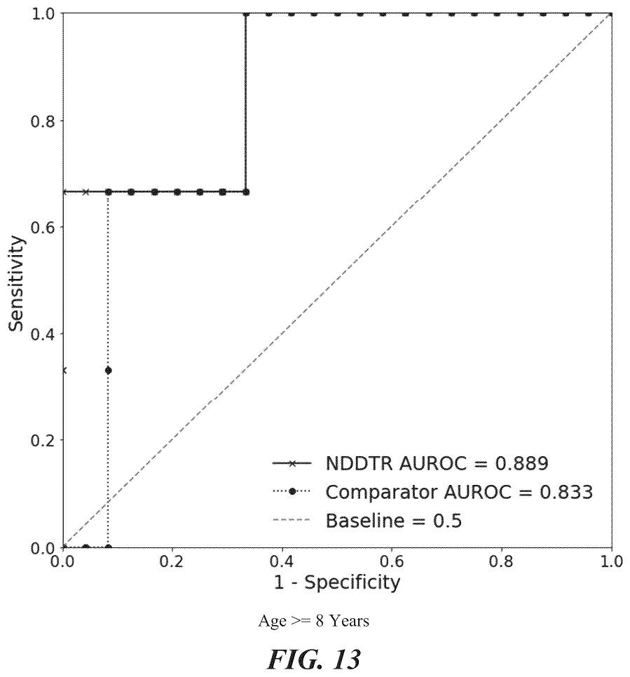

FIGS. 11, 12, and 13 display ROC curves illustrating operation of the NDDTR model. Particularly, the ROC curves of FIGS. 11, 12, and 13 correspond to each of the three groups shown in Table 3, more particularly, age groups <5 years, 5 to <8 years, >=8 years, respectively.

Overall, the data in the Examples demonstrates that the NDDTR model can be employed with different datasets and can be used to develop and provide a therapy recommendation.

### Additional Embodiments

The following additional embodiments provide further examples of the subject matter disclosed herein.

A 1^{st} embodiment is a method implemented via a computing device, the method comprising receiving, by the computing device, data associated with a subject having a neurodevelopmental disorder (NDD), the data associated with the subject comprising demographic data, schooling data, family medical data, prior therapy data, observational assessment data, medication data, goals data, or combinations thereof, and evaluating, by the computing device, the data associated with the subject via a neurodevelopmental disorder treatment recommendation (NDDTR) model, wherein the NDDTR model is configured to evaluate the data associated with the subject to determine a therapy recommendation, wherein the therapy recommendation comprises a standard of care.

A 2^{nd} embodiment is the method of the 1^{st} embodiment, wherein the neurodevelopmental disorder is autism spectrum disorder (ASD).

A 3^{rd} embodiment is the method of one of the 1^{st} through the 2^{nd} embodiments, wherein the data associated with the subject comprise the demographic data, wherein the demographic data comprise age data.

A 4^{th} embodiment is the method of one of the 1^{st} through the 2^{nd} embodiments, wherein the data associated with the subject comprise the schooling data, wherein the schooling data comprise an indication of whether the subject attends school, an indication of whether the subject has been assigned a school aide, an indication of whether the subject is a part of a special education program, or combinations thereof.

A 5^{th} embodiment is the method of one of the 1^{st} through the 4^{th} embodiments, wherein the data associated with the subject comprise the family medical data, wherein the family medical data comprise an indication as to the presence or absence of depression or manic-depression in an immediate family member, an indication as to the presence or absence of substance abuse or dependence in an immediate family member, an indication as to the presence or absence of an anxiety disorder in an immediate family member, an indication as to the presence or absence of attention deficit hyperactivity disorder (ADHD) in an immediate family member, or combinations thereof.

A 6^{th} embodiment is the method of one of the 1^{st} through the 5^{th} embodiments, wherein the data associated with the subject comprise the prior therapy data, wherein the prior therapy data comprise an indication of the subject having previously received occupational therapy, an indication of the subject having previously received speech therapy, an indication of duration of applied behavioral analysis (ABA) therapy previously received by the subject, an indication of amount of ABA therapy previously received by the subject, or combinations thereof.

A 7^{th} embodiment is the method of one of the 1^{st} through the 6^{th} embodiments, wherein the data associated with the subject comprise an indication of the subject's tendency toward aggressive behavior, an indication of the subject's tendency toward stereotypy, an indication of the subject's tendency toward destructive behaviors, an indication of the consequences implemented by a caregiver of the subject responsive to negative behavior, an indication of the subject's ability to be understood, an indication of the subject's ability to understand others, an indication of variety of foods eaten by the subject, an indication of the subject's ability use a toilet independently, an indication of the subject's ability to bathe independently, an indication of stimulatory behaviors exhibited by the subject, or combinations thereof.

An 8^{th} embodiment is the method of one of the 1^{st} through the 7^{th} embodiments, wherein the data associated with the subject comprise the medication data, wherein the medication data comprise an indication of a medication for sleep used by the subject, an indication of a medication for allergies used by the subject, or combinations thereof.

A 9^{th} embodiment is the method of one of the 1^{st} through the 8^{th} embodiments, wherein the data associated with the subject comprise the goals data, wherein the goals data comprise an indication of a goal of improved communication skills, an indication of a goal of improved diet, an indication of a goal of increased independence, an indication of a goal of improved ability to express emotions, of combinations thereof.

A 10^{th} embodiment is the method of one of the 1^{st} through the 9^{th} embodiments, wherein the data associated with the subject comprise a plurality of data features, wherein the plurality of data features comprises not more than about 30 different data features.

An 11^{th} embodiment is the method of one of the 1^{st} through the 10^{th} embodiments, wherein the data associated with the subject comprise structured data.

A 12^{th} embodiment is the method of one of the 1^{st} through the 11^{th} embodiments, wherein the NDDTR model is a machine learning model selected from the group consisting of a deep learning model, a generative adversarial network model, a computational neural network model, a recurrent neural network model, a perceptron model, a classical tree-based machine learning model, a decision tree type model, a regression type model, a classification model, a reinforcement learning model, and combinations thereof.

A 13^{th} embodiment is the method of the 12^{th} embodiment, wherein the machine learning model is a gradient-boosted tree model.

A 14^{th} embodiment is the method of the 13^{th} embodiment, wherein the gradient-boosted tree model comprises a plurality of decision trees.

A 15^{th} embodiment is the method of one of the 13^{th} through the 14^{th} embodiments, wherein the gradient-boosted tree model comprises from about 50 decision trees to about 400 decision trees.

A 16^{th} embodiment is the method of one of the 14^{th} through the 15^{th} embodiments, wherein the plurality of decision trees are weighted.

A 17^{th} embodiment is the method of one of the 12^{th} through the 16^{th} embodiments, further comprising identifying NDDTR model hyperparameters, wherein the NDDTR model hyperparameters comprise tree depth, number of decision trees, learning rate, scale positive weight, alpha regularization parameter, gamma regularization parameter, or combinations thereof, and tuning the NDDTR model hyperparameters, wherein the tuning of the NDDTR model hyperparameters is effective to provide for an NDDTR model sensitivity of from about 0.75 to about 0.99.

An 18^{th} embodiment is the method of one of the 1^{st} through the 17^{th} embodiments, further comprising transforming the data associated with the subject into discrete numerical vectors, wherein the discrete numerical vectors are provided to the NDDTR model to determine the therapy recommendation.

A 19^{th} embodiment is the method of one of the 1^{st} through the 18^{th} embodiments, wherein the standard of care comprises an indication of intensity of therapy.

A 20^{th} embodiment is the method of one of the 1^{st} through the 19^{th} embodiments, wherein the standard of care comprises an indication of services.

A 21^{st} embodiment is the method of one of the 1^{st} through the 20^{th} embodiments, wherein the standard of care comprises an indication of one of a comprehensive therapy or a focused therapy.

A 22^{nd} embodiment is the method of one of the 1^{st} through the 21st embodiments, further comprising providing therapy to the subject based upon the therapy recommendation.

A 23^{rd} embodiment is the method of the 22^{nd} embodiment, wherein the therapy is provided to the subject via the computing device, a second computing device in signal communication with the computing device, or combinations thereof.

A 24^{th} embodiment is the method of one of the 22^{nd} through the 23^{rd} embodiments, wherein the therapy comprises ABA therapy.

A 25^{th} embodiment is the method of one of the 1^{st} through the 24^{th} embodiments, wherein the computing device comprises an edge computing device, a cloud computing device, or both.

A 26^{th} embodiment is the method of one of the 1^{st} through the 25^{th} embodiments, wherein the data associated with the subject comprise the demographic data, wherein the demographic data comprise age data, intelligence quotient (IQ) data, sex data, handedness data, race data, ethnicity data, socioeconomic status data, financial data, monetary income data, monetary savings data, parental and/or custodial employment data, parental and/or custodial education data, health insurance data, health insurance provider data, or combinations thereof.

A 27^{th} embodiment is the method of one of the 1^{st} through the 26^{th} embodiments, wherein the data associated with the subject comprise the schooling data, wherein the schooling data comprise an indication of whether the subject attends school, an indication of whether the subject has been assigned a school aide, an indication of whether the subject is a part of a special education program, school grade data, an indication of whether the subject receives any special school services, an indication of whether the subject receives additional services as part of a special education program, or combinations thereof.

A 28^{th} embodiment is the method of one of the 1^{st} through the 27^{th} embodiments, wherein the data associated with the subject comprise the family medical data, wherein the family medical data comprise an indication as to the presence or absence of depression or manic-depression in an immediate family member, an indication as to the presence or absence of substance abuse or dependence in an immediate family member, an indication as to the presence or absence of an anxiety disorder in an immediate family member, an indication as to the presence or absence of ADHD in an immediate family member, an indication as to the presence or absence of a learning disability in an immediate family member, an indication as to the presence or absence of psychosis or schizophrenia in an immediate family member, or combinations thereof.

A 29^{th} embodiment is the method of one of the 1^{st} through the 28^{th} embodiments, wherein the data associated with the subject comprise the prior therapy data; wherein the prior therapy data comprise an indication of the subject having previously received ABA therapy; an indication of the subject having previously received occupational therapy; an indication of the subject having previously received speech therapy; an indication of type of ABA therapy previously received by the subject; an indication of duration of ABA therapy previously received by the subject; an indication of amount of ABA therapy previously received by the subject; an indication of the subject having previously received physical therapy; an indication of the subject having previously received any therapy other than ABA therapy, speech therapy, occupational therapy, or physical therapy; or combinations thereof.

A 30^{th} embodiment is the method of one of the 1^{st} through the 29^{th} embodiments, wherein the data associated with the subject comprise an indication of the subject's tendency toward aggressive behavior, an indication of the frequency and/or severity of the subject's aggressive behavior, an indication of the subject's tendency toward engaging in self-injury behavior, an indication of the frequency and/or severity of the subject's self-injury behavior, an indication of the subject's tendency toward stereotypy, an indication of the frequency and/or severity of the subject's stereotypy, an indication of the subject's tendency toward destructive behaviors, an indication of the frequency and/or severity of the subject's destructive behaviors, an indication of the frequency and/or severity of the subject's destruction of property, an indication of the consequences implemented by a caregiver of the subject responsive to negative behavior, an indication of the subject's ability to be understood, an indication of the subject's ability to understand others, an indication of variety of foods eaten by the subject, an indication of the subject's ability use a toilet independently, an indication of the subject's ability to bathe independently, an indication of stimulatory behaviors exhibited by the subject, an indication of whether the subject can be described as easy-going going with the flow, an indication of whether the subject is anxious or easily upset by things that would not regularly upset others in otherwise similar circumstances, an indication of whether the subject follows simple directions in a home setting, or combinations thereof.

A 31^{st} embodiment is the method of one of the 1^{st} through the 30^{th} embodiments, wherein the data associated with the subject comprise the medication data, wherein the medication data comprise an indication of any medication used by the subject, wherein the medication data comprise an indication of a medication for sleep used by the subject, an indication of a medication for allergies used by the subject, an indication of a medication for ASD used by the subject, an indication of a medication for ADHD used by the subject, an indication of a medication for anxiety used by the subject, an indication of a medication for depression used by the subject, an indication of a medication for a behavior or mood related condition used by the subject, or combinations thereof.

A 32^{nd} embodiment is the method of one of the 1^{st} through the 31^{st} embodiments, wherein the data associated with the subject comprise the goals data, wherein the goals data comprise an indication of a goal of improved communication skills, an indication of a goal of improved diet, an indication of a goal of increased independence, an indication of a goal of improved ability to express emotions, an indication of a goal of improved social skills, an indication of a goal of improved ability to participate in family activities, an indication of a goal of decreased challenging behaviors, an indication of a goal of getting along better with parents and/or siblings, an indication of a goal of learning toilet training, an indication of a goal of learning new ways to leave non-preferred activities, an indication of a goal of doing what they are told without responding inappropriately, an indication of a goal of keeping their body and others around them safe, an indication of a goal of increased participation in general education classrooms or settings, an indication of a goal of increased flexibility and/or being less rigid, of combinations thereof.

A 33^{rd} embodiment is the method of one of the 1^{st} through the 31^{st} embodiments, wherein the data associated with the subject further comprise diagnosis data, sleep and wake patterns data, a self-stimulatory behaviors data, restrictive and repetitive behaviors data, communication skills data, social skills data, or combinations thereof.

A 34^{th} embodiment is the method of one of the 1^{st} through the 33^{rd} embodiments, wherein the data associated with the subject comprise a plurality of data features, wherein the plurality of data features comprises from about 12 to about 30 different data features.

A 35^{th} embodiment is the method of one of the 1^{st} through the 34^{th} embodiments, wherein the data associated with the subject comprise a plurality of data features, wherein the plurality of data features comprises from about 24 to about 30 different data features.

A 36^{th} embodiment is the method of one of the 13^{th} through the 15^{th} embodiments, wherein the gradient-boosted tree model comprises from about 50 decision trees to about 300 decision trees.

A 37^{th} embodiment is the method of one of the 13^{th} through the 15^{th} embodiments, wherein the gradient-boosted tree model comprises from about 50 decision trees to about 150 decision trees.

A 38^{th} embodiment is the method of one of the 17^{th} through the 37^{th} embodiments, wherein the NDDTR model has a tree depth of at least 2 and not more than 6.

A 39^{th} embodiment is the method of one of the 17^{th} through the 38^{th} embodiments, wherein the NDDTR model has a tree depth of not more than 5.

A 40^{th} embodiment is the method of one of the 17^{th} through the 39^{th} embodiments, wherein the NDDTR model has a tree depth of not more than 3.

A 41^{st} embodiment is the method of one of the 17^{th} through 40^{th} the embodiments, wherein the NDDTR model has a learning rate of equal to or less than about 0.4.

A 42^{nd} embodiment is the method of one of the 17^{th} through the 41^{st} embodiments, wherein the NDDTR model has a scale positive weight of from about 0.1 to about 10.

A 43^{rd} embodiment is the method of one of the 17^{th} through the 42^{nd} embodiments, wherein the NDDTR model has an alpha regularization parameter of from about 0 to about 1.

A 44^{th} embodiment is the method of one of the 17^{th} through the 43^{rd} embodiments, wherein the NDDTR model has a gamma regularization parameter of from about 0 to about 1.

A 45^{th} embodiment is the method of one of the 1^{st} through the 44^{th} embodiments, wherein the therapy recommendation is delivered to the subject and/or a caregiver thereof via the computing device, a second computing device in signal communication with the computing device, or combinations thereof.

A 46^{th} embodiment is the method of one of the 22^{nd} through the 23^{rd} embodiments, wherein the therapy comprises ABA therapy, speech therapy, positive reinforcement therapy, behavioral management therapy, play therapy, cognitive behavioral therapy, joint attention therapy, nutritional therapy, occupational therapy, parent-mediated therapy, physical therapy, social skills training therapy, or combinations thereof.

A 47^{th} embodiment is a computing system, the system comprising a computing device, the computing device comprising a processor and a non-transitory computer-readable medium, wherein the non-transitory computer-readable medium includes instructions configured to cause the processor to implement an NDDTR model, wherein the NDDTR model, when implemented via the processor, causes the computing device to receive data associated with a subject having an NDD, the data associated with the subject comprising demographic data, schooling data, family medical data, prior therapy data, observational assessment data, medication data, goals data, or combinations thereof; and evaluate the data associated with the subject via the NDDTR model, wherein the NDDTR model is configured to evaluate the data associated with the subject to determine a therapy recommendation, wherein the therapy recommendation comprises a standard of care.

A 48^{th} embodiment is the computing system of the 47^{th} embodiment, wherein the neurodevelopmental disorder is ASD.

A 49^{th} embodiment is the computing system of one of the 47^{th} through the 48^{th} embodiments, wherein the data associated with the subject comprise the demographic data, wherein the demographic data comprise age data.

A 50^{th} embodiment is the computing system of one of the 47^{th} through the 49^{th} embodiments, wherein the data associated with the subject comprise the schooling data, wherein the schooling data comprise an indication of whether the subject attends school, an indication of whether the subject has been assigned a school aide, an indication of whether the subject is a part of a special education program, or combinations thereof.

A 51^{st} embodiment is the computing system of one of the 47^{th} through the 50^{th} embodiments, wherein the data associated with the subject comprise the family medical data, wherein the family medical data comprise an indication as to the presence or absence of depression or manic-depression in an immediate family member, an indication as to the presence or absence of substance abuse or dependence in an immediate family member, an indication as to the presence or absence of an anxiety disorder in an immediate family member, an indication as to the presence or absence of ADHD in an immediate family member, or combinations thereof.

A 52^{nd} embodiment is the computing system of one of the 47^{th} through the 51^{st} embodiments, wherein the data associated with the subject comprise the prior therapy data, wherein the prior therapy data comprise an indication of the subject having previously received occupational therapy, an indication of the subject having previously received speech therapy, an indication of duration of ABA therapy previously received by the subject, an indication of amount of ABA therapy previously received by the subject, or combinations thereof.

A 53^{rd} embodiment is the computing system of one of the 47^{th} through the 52^{nd} embodiments, wherein the data associated with the subject comprise an indication of the subject's tendency toward aggressive behavior, an indication of the subject's tendency toward stereotypy, an indication of the subject's tendency toward destructive behaviors, an indication of the consequences implemented by a caregiver of the subject responsive to negative behavior, an indication of the subject's ability to be understood, an indication of the subject's ability to understand others, an indication of variety of foods eaten by the subject, an indication of the subject's ability use a toilet independently, an indication of the subject's ability to bathe independently, an indication of stimulatory behaviors exhibited by the subject, or combinations thereof.

A 54^{th} embodiment is the computing system of one of the 47^{th} through the 53^{rd} embodiments, wherein the data associated with the subject comprise the medication data, wherein the medication data comprise an indication of a medication for sleep used by the subject, an indication of a medication for allergies used by the subject, or combinations thereof.

A 55^{th} embodiment is the computing system of one of the 47^{th} through the 54^{th} embodiments, wherein the data associated with the subject comprise the goals data, wherein the goals data comprise an indication of a goal of improved communication skills, an indication of a goal of improved diet, an indication of a goal of increased independence, an indication of a goal of improved ability to express emotions, of combinations thereof.

A 56^{th} embodiment is the computing system of one of the 47^{th} through the 55^{th} embodiments, wherein the data associated with the subject comprise a plurality of data features, wherein the plurality of data features comprises not more than about 30 different data features.

A 57^{th} embodiment is the computing system of one of the 47^{th} through the 56^{th} embodiments, wherein the data associated with the subject comprise structured data.

A 58^{th} embodiment is the computing system of one of the 47^{th} through the 57^{th} embodiments, wherein the NDDTR model is a machine learning model selected from the group consisting of a deep learning model, a generative adversarial network model, a computational neural network model, a recurrent neural network model, a perceptron model, a classical tree-based machine learning model, a decision tree type model, a regression type model, a classification model, a reinforcement learning model, and combinations thereof.

A 59^{th} embodiment is the computing system of the 58^{th} embodiment, wherein the machine learning model is a gradient-boosted tree model.

A 60^{th} embodiment is the computing system of the 59^{th} embodiment, wherein the gradient-boosted tree model comprises a plurality of decision trees.

A 61^{st} embodiment is the computing system of one of the 59^{th} through the 60^{th} embodiments, wherein the gradient-boosted tree model comprises from about 50 decision trees to about 400 decision trees.

A 62^{nd} embodiment is the computing system of one of the 60^{th} through the 61^{st} embodiments, wherein the plurality of decision trees are weighted.

A 63^{rd} embodiment is the computing system of one of the 58^{th} through the 62^{nd} embodiments, further comprising identifying NDDTR model hyperparameters, wherein the NDDTR model hyperparameters comprise tree depth, number of decision trees, learning rate, scale positive weight, alpha regularization parameter, gamma regularization parameter, or combinations thereof, and tuning the NDDTR model hyperparameters, wherein the tuning of the NDDTR model hyperparameters is effective to provide for an NDDTR model sensitivity of from about 0.75 to about 0.99.

A 64^{th} embodiment is the computing system of one of the 47^{th} through the 63^{rd} embodiments, further comprising transforming the data associated with the subject into discrete numerical vectors, wherein the discrete numerical vectors are provided to the NDDTR model to determine the therapy recommendation.

A 65^{th} embodiment is the computing system of one of the 47^{th} through the 64^{th} embodiments, wherein the standard of care comprises an indication of intensity of therapy.

A 66^{th} embodiment is the computing system of one of the 47^{th} through the 65^{th} embodiments, wherein the standard of care comprises an indication of services.

A 67^{th} embodiment is the computing system of one of the 47^{th} through the 66^{th} embodiments, wherein the standard of care comprises an indication of one of a comprehensive therapy or a focused therapy.

A 68^{th} embodiment is the computing system of one of the 47^{th} through the 67^{th} embodiments, further comprising providing therapy to the subject based upon the therapy recommendation.

A 69^{th} embodiment is the computing system of the 68^{th} embodiment, wherein the therapy is provided to the subject via the computing device, a second computing device in signal communication with the computing device, or combinations thereof.

A 70^{th} embodiment is the computing system of one of the 68^{th} through the 69^{th} embodiments, wherein the therapy comprises ABA therapy.

A 71^{st} embodiment is the computing system of one of the 47^{th} through the 70^{th} embodiments, wherein the computing device comprises an edge computing device, a cloud computing device, or both.

A 72^{nd} embodiment is the computing system of one of the 47^{th} through the 71^{st} embodiments, wherein the data associated with the subject comprise the demographic data, wherein the demographic data comprise age data, IQ data, sex data, handedness data, race data, ethnicity data, socioeconomic status data, financial data, monetary income data, monetary savings data, parental and/or custodial employment data, parental and/or custodial education data, health insurance data, health insurance provider data, or combinations thereof.

A 73^{rd} embodiment is the computing system of one of the 47^{th} through the 72^{nd} embodiments, wherein the data associated with the subject comprise the schooling data, wherein the schooling data comprise an indication of whether the subject attends school, an indication of whether the subject has been assigned a school aide, an indication of whether the subject is a part of a special education program, school grade data, an indication of whether the subject receives any special school services, an indication of whether the subject receives additional services as part of a special education program, or combinations thereof.

A 74^{th} embodiment is the computing system of one of the 47^{th} through the 73^{rd} embodiments, wherein the data associated with the subject comprise the family medical data, wherein the family medical data comprise an indication as to the presence or absence of depression or manic-depression in an immediate family member, an indication as to the presence or absence of substance abuse or dependence in an immediate family member, an indication as to the presence or absence of an anxiety disorder in an immediate family member, an indication as to the presence or absence of ADHD in an immediate family member, an indication as to the presence or absence of a learning disability in an immediate family member, an indication as to the presence or absence of psychosis or schizophrenia in an immediate family member, or combinations thereof.

A 75^{th} embodiment is the computing system of one of the 47^{th} through the 74^{th} embodiments, wherein the data associated with the subject comprise the prior therapy data; wherein the prior therapy data comprise an indication of the subject having previously received ABA therapy; an indication of the subject having previously received occupational therapy; an indication of the subject having previously received speech therapy; an indication of type of ABA therapy previously received by the subject; an indication of duration of ABA therapy previously received by the subject; an indication of amount of ABA therapy previously received by the subject; an indication of the subject having previously received physical therapy; an indication of the subject having previously received any therapy other than ABA therapy, speech therapy, occupational therapy, or physical therapy; or combinations thereof.

A 76^{th} embodiment is the computing system of one of the 47^{th} through the 75^{th} embodiments, wherein the data associated with the subject comprise an indication of the subject's tendency toward aggressive behavior, an indication of the frequency and/or severity of the subject's aggressive behavior, an indication of the subject's tendency toward engaging in self-injury behavior, an indication of the frequency and/or severity of the subject's self-injury behavior, an indication of the subject's tendency toward stereotypy, an indication of the frequency and/or severity of the subject's stereotypy, an indication of the subject's tendency toward destructive behaviors, an indication of the frequency and/or severity of the subject's destructive behaviors, an indication of the frequency and/or severity of the subject's destruction of property, an indication of the consequences implemented by a caregiver of the subject responsive to negative behavior, an indication of the subject's ability to be understood, an indication of the subject's ability to understand others, an indication of variety of foods eaten by the subject, an indication of the subject's ability use a toilet independently, an indication of the subject's ability to bathe independently, an indication of stimulatory behaviors exhibited by the subject, an indication of whether the subject can be described as easy-going going with the flow, an indication of whether the subject is anxious or easily upset by things that would not regularly upset others in otherwise similar circumstances, an indication of whether the subject follows simple directions in a home setting, or combinations thereof.

A 77^{th} embodiment is the computing system of one of the 47^{th} through the 76^{th} embodiments, wherein the data associated with the subject comprise the medication data, wherein the medication data comprise an indication of a medication for sleep used by the subject, an indication of a medication for allergies used by the subject, an indication of a medication for ASD used by the subject, an indication of a medication for ADHD used by the subject, an indication of a medication for anxiety used by the subject, an indication of a medication for depression used by the subject, an indication of a medication for a behavior or mood related condition used by the subject, or combinations thereof.

A 78^{th} embodiment is the computing system of one of the 47^{th} through the 77^{th} embodiments, wherein the data associated with the subject comprise the goals data, wherein the goals data comprise an indication of a goal of improved communication skills, an indication of a goal of improved diet, an indication of a goal of increased independence, an indication of a goal of improved ability to express emotions, an indication of a goal of improved social skills, an indication of a goal of improved ability to participate in family activities, an indication of a goal of decreased challenging behaviors, an indication of a goal of getting along better with parents and/or siblings, an indication of a goal of learning toilet training, an indication of a goal of learning new ways to leave non-preferred activities, an indication of a goal of doing what they are told without responding inappropriately, an indication of a goal of keeping their body and others around them safe, an indication of a goal of increased participation in general education classrooms or settings, an indication of a goal of increased flexibility and/or being less rigid, of combinations thereof.

A 79^{th} embodiment is the computing system of one of the 47^{th} through the 78^{th} embodiments, wherein the data associated with the subject further comprise diagnosis data, sleep and wake patterns data, a self-stimulatory behaviors data, restrictive and repetitive behaviors data, communication skills data, social skills data, or combinations thereof.

An 80^{th} embodiment is the computing system of one of the 47^{th} through the 79^{th} embodiments, wherein the data associated with the subject comprise a plurality of data features, wherein the plurality of data features comprises from about 12 to about 30 different data features.

An 81^{st} embodiment is the computing system of one of the 47^{th} through the 80^{th} embodiments, wherein the data associated with the subject comprise a plurality of data features, wherein the plurality of data features comprises from about 24 to about 30 different data features.

An 82^{nd} embodiment is the computing system of one of the 59^{th} through the 61^{st} embodiments, wherein the gradient-boosted tree model comprises from about 50 decision trees to about 300 decision trees.

An 83^{rd} embodiment is the computing system of one of the 59^{th} through the 61^{st} embodiments, wherein the gradient-boosted tree model comprises from about 50 decision trees to about 150 decision trees.

An 84^{th} embodiment is the computing system of one of the 47^{th} through the 83^{rd} embodiments, wherein the NDDTR model has a tree depth of at least 2 and not more than 6.

An 85^{th} embodiment is the computing system of one of the 47^{th} through the 84^{th} embodiments, wherein the NDDTR model has a tree depth of not more than 5.

An 86^{th} embodiment is the computing system of one of the 47^{th} through the 85^{th} embodiments, wherein the NDDTR model has a tree depth of not more than 3.

An 87^{th} embodiment is the computing system of one of the 47^{th} through the 86^{th} embodiments, wherein the NDDTR model has a learning rate of equal to or less than about 0.4.

An 88^{th} embodiment is the computing system of one of the 47^{th} through the 87^{th} embodiments, wherein the NDDTR model has a scale positive weight of from about 0.1 to about 10.

An 89^{th} embodiment is the computing system of one of the 47^{th} through the 88^{th} embodiments, wherein the NDDTR model has an alpha regularization parameter of from about 0 to about 1.

A 90^{th} embodiment is the computing system of one of the 47^{th} through the 89^{th} embodiments, wherein the NDDTR model has a gamma regularization parameter of from about 0 to about 1.

A 91^{st} embodiment is the computing system of one of the 47^{th} through the 90^{th} embodiments, wherein the computing system optionally comprises a second computing device in signal communication with the computing device; wherein the therapy recommendation is delivered to the subject and/or a caregiver thereof via the computing device and/or the second computing device.

A 92^{nd} embodiment is the computing system of one of the 62^{nd} through the 63^{rd} embodiments, wherein the therapy comprises ABA therapy, speech therapy, positive reinforcement therapy, behavioral management therapy, play therapy, cognitive behavioral therapy, joint attention therapy, nutritional therapy, occupational therapy, parent-mediated therapy, physical therapy, social skills training therapy, or combinations thereof.

A 93^{rd} embodiment is a method implemented via a computing device, the method comprising receiving, by the computing device, training data associated with a plurality of subjects, wherein at least a portion of the subjects are individuals characterized as having a NDD, and wherein the training data associated with each of the plurality of subjects comprise demographic data, schooling data, family medical data, prior therapy data, observational assessment data, medication data, goals data, or combinations thereof, and processing the training data associated with the plurality of subjects to yield an NDDTR model, wherein the NDDTR model is configured to evaluate data associated with a subject to evaluate the data associated with the subject to determine a therapy recommendation, wherein the therapy recommendation comprises a standard of care.

A 94^{th} embodiment is the method of the 93^{rd} embodiment, wherein the neurodevelopmental disorder is ASD.

A 95^{th} embodiment is the method of one of the 93^{rd} through the 94^{th} embodiments, wherein the data associated with the plurality of subjects comprise the demographic data, wherein the demographic data comprise age data.

A 96^{th} embodiment is the method of one of the 93^{rd} through the 95^{th} embodiments, wherein the data associated with the plurality of subjects comprise the schooling data, wherein the schooling data comprise an indication of whether one or more of the plurality of subjects attends school, an indication of whether one or more of the plurality of subjects has been assigned a school aide, an indication of whether one or more of the plurality of subjects is a part of a special education program, or combinations thereof.

A 97^{th} embodiment is the method of one of the 93^{rd} through the 96^{th} embodiments, wherein the data associated with the plurality of subjects comprise the family medical data, wherein the family medical data comprise an indication as to the presence or absence of depression or manic-depression in an immediate family member of one or more of the plurality of subjects, an indication as to the presence or absence of substance abuse or dependence in an immediate family member of one or more of the plurality of subjects, an indication as to the presence or absence of an anxiety disorder in an immediate family member of one or more of the plurality of subjects, an indication as to the presence or absence of ADHD in an immediate family member of one or more of the plurality of subjects, or combinations thereof.

A 98^{th} embodiment is the method of one of the 93^{rd} through the 97^{th} embodiments, wherein the data associated with the plurality of subjects comprise the prior therapy data, wherein the prior therapy data comprise an indication of one or more of the plurality of subjects having previously received occupational therapy, an indication of one or more of the plurality of subjects having previously received speech therapy, an indication of duration of ABA therapy previously received by one or more of the plurality of subjects, an indication of amount of ABA therapy previously received by one or more of the plurality of subjects, or combinations thereof.

A 99^{th} embodiment is the method of one of the 93^{rd} through the 98^{th} embodiments, wherein the data associated with the plurality of subjects comprise an indication of one or more of the plurality of subject's tendency toward aggressive behavior, an indication of one or more of the plurality of subject's tendency toward stereotypy, an indication of one or more of the plurality of subject's tendency toward destructive behaviors, an indication of the consequences implemented by a caregiver of the one or more of the plurality of subjects responsive to negative behavior, an indication of one or more of the plurality of subject's ability to be understood, an indication of one or more of the plurality of subject's ability to understand others, an indication of variety of foods eaten by one or more of the plurality of subjects, an indication of one or more of the plurality of subject's ability use a toilet independently, an indication of one or more of the plurality of subject's ability to bathe independently, an indication of stimulatory behaviors exhibited by one or more of the plurality of subjects, or combinations thereof.

A 100^{th} embodiment is the method of one of the 93^{rd} through the 99^{th} embodiments, wherein the data associated with the plurality of subjects comprise the medication data, wherein the medication data comprise an indication of a medication for sleep used by one or more of the plurality of subjects, an indication of a medication for allergies used by one or more of the plurality of subjects, or combinations thereof.

A 101^{st} embodiment is the method of one of the 93^{rd} through the 100^{th} embodiments, wherein the data associated with the plurality of subjects comprise the goals data, wherein the goals data comprise an indication of a goal of improved communication skills, an indication of a goal of improved diet, an indication of a goal of increased independence, an indication of a goal of improved ability to express emotions, of combinations thereof.

A 102^{nd} embodiment is the method of one of the 93^{rd} through the 101^{th} embodiments, wherein the data associated with each of the plurality of subjects comprise a plurality of data features, wherein the plurality of data features comprises not more than 30 different data features.

A 103^{rd} embodiment is the method of one of the 93^{rd} through the 102^{nd} embodiments, wherein the data associated with the plurality of subjects comprise structured data.

A 104^{th} embodiment is the method of one of the 93^{rd} through the 103^{th} embodiments, wherein the NDDTR model is a machine learning model selected from the group consisting of a deep learning model, a generative adversarial network model, a computational neural network model, a recurrent neural network model, a perceptron model, a classical tree-based machine learning model, a decision tree type model, a regression type model, a classification model, a reinforcement learning model, and combinations thereof.

A 105^{th} embodiment is the method of the 104^{th} embodiment, wherein the machine learning model is a gradient-boosted tree model.

A 106^{th} embodiment is the method of the 105^{th} embodiments, wherein the gradient-boosted tree model comprises a plurality of decision trees.

A 107^{th} embodiment is the method of one of the 104^{th} through the 105^{th} embodiments, wherein the gradient-boosted tree model comprises from about 50 decision trees to about 400 decision trees.

A 108^{th} embodiment is the method of one of the 106^{th} through the 107^{th} embodiments, wherein the plurality of decision trees are weighted.

A 109^{th} embodiment is the method of one of the 104^{th} through the 108^{th} embodiments, further comprising identifying NDDTR model hyperparameters, wherein the NDDTR model hyperparameters comprise tree depth, number of decision trees, learning rate, scale positive weight, alpha regularization parameter, gamma regularization parameter, or combinations thereof, and tuning the NDDTR model hyperparameters, wherein the tuning of the NDDTR model hyperparameters is effective to provide for an NDDTR model sensitivity of from about 0.75 to about 0.99.

A 110^{th} embodiment is the method of the 109^{th} embodiment, wherein the NDDTR model hyperparameters comprise a maximum tree depth of from 2 to 3.

A 111^{th} embodiment is the method of one of the 109^{th} through the 110^{th} embodiments, wherein the NDDTR model hyperparameters comprise a number of decision trees of from about 75 to about 125.

A 112^{th} embodiment is the method of one of the 109^{th} through the 111^{th} embodiments, wherein the NDDTR model hyperparameters comprise a scale positive weight of less than about 0.3.

A 113^{th} embodiment is the method of one of the 93^{rd} through the 112^{th} embodiments, further comprising transforming the data associated with each of the plurality of subjects into discrete numerical vectors, wherein the discrete numerical vectors are provided to the NDDTR model to determine the therapy recommendation.

A 114^{th} embodiment is the method of one of the 93^{rd} through the 113^{th} embodiments, wherein the standard of care comprises an indication of intensity of therapy.

A 115^{th} embodiment is the method of one of the 93^{rd} through the 114^{th} embodiments, wherein the standard of care comprises an indication of services.

A 116^{th} embodiment is the method of one of the 93^{rd} through the 115^{th} embodiments, wherein the standard of care comprises an indication of one of a comprehensive therapy or a focused therapy.

A 117^{th} embodiment is the method of one of the 93^{rd} through the 116^{th} embodiments, wherein the computing device comprises an edge computing device, a cloud computing device, or both.

A 118^{th} embodiment is the method of one of the 93^{rd} through the 117^{th} embodiments, wherein the data associated with the plurality of subjects comprise the demographic data, wherein the demographic data comprise age data, IQ data, sex data, handedness data, race data, ethnicity data, socioeconomic status data, financial data, monetary income data, monetary savings data, parental and/or custodial employment data, parental and/or custodial education data, health insurance data, health insurance provider data, or combinations thereof.

A 119^{th} embodiment is the method of one of the 93^{rd} through the 118^{th} embodiments, wherein the data associated with the plurality of subjects comprise the schooling data, wherein the schooling data comprise an indication of whether one or more of the plurality of subjects attends school, an indication of whether one or more of the plurality of subjects has been assigned a school aide, an indication of whether one or more of the plurality of subjects is a part of a special education program, school grade data, an indication of whether one or more of the plurality of subjects receives any special school services, an indication of whether one or more of the plurality of subjects receives additional services as part of a special education program, or combinations thereof.

A 120^{th} embodiment is the method of one of the 93^{rd} through the 119^{th} embodiments, wherein the data associated with the plurality of subjects comprise the family medical data, wherein the family medical data comprise an indication as to the presence or absence of depression or manic-depression in an immediate family member of one or more of the plurality of subjects, an indication as to the presence or absence of substance abuse or dependence in an immediate family member of one or more of the plurality of subjects, an indication as to the presence or absence of an anxiety disorder in an immediate family member of one or more of the plurality of subjects, an indication as to the presence or absence of ADHD in an immediate family member of one or more of the plurality of subjects, an indication as to the presence or absence of a learning disability in an immediate family member of one or more of the plurality of subjects, an indication as to the presence or absence of psychosis or schizophrenia in an immediate family member of one or more of the plurality of subjects, or combinations thereof.

A 121^{st} embodiment is the method of one of the 93^{rd} through the 120^{th} embodiments, wherein the data associated with the plurality of subjects comprise the prior therapy data; wherein the prior therapy data comprise an indication of one or more of the plurality of subjects having previously received ABA therapy; an indication of one or more of the plurality of subjects having previously received occupational therapy; an indication of one or more of the plurality of subjects having previously received speech therapy; an indication of type of ABA therapy previously received by one or more of the plurality of subjects; an indication of duration of ABA therapy previously received by one or more of the plurality of subjects; an indication of amount of ABA therapy previously received by one or more of the plurality of subjects; an indication of one or more of the plurality of subjects having previously received physical therapy; an indication of one or more of the plurality of subjects having previously received any therapy other than ABA therapy, speech therapy, occupational therapy, or physical therapy; or combinations thereof.

A 122^{nd} embodiment is the method of one of the 93^{rd} through the 121^{st} embodiments, wherein the data associated with the plurality of subjects comprise an indication of one or more of the plurality of subject's tendency toward aggressive behavior, an indication of the frequency and/or severity of one or more of the plurality of subject's aggressive behavior, an indication of one or more of the plurality of subject's tendency toward self-injury behavior, an indication of the frequency and/or severity of one or more of the plurality of subject's self-injury behavior, an indication of one or more of the plurality of subject's tendency toward stereotypy, an indication of the frequency and/or severity of one or more of the plurality of subject's stereotypy, an indication of one or more of the plurality of subject's tendency toward destructive behaviors, an indication of the frequency and/or severity of one or more of the plurality of subject's destructive behaviors, an indication of the frequency and/or severity of one or more of the plurality of subject's destruction of property, an indication of the consequences implemented by a caregiver of the one or more of the plurality of subjects responsive to negative behavior, an indication of one or more of the plurality of subject's ability to be understood, an indication of one or more of the plurality of subject's ability to understand others, an indication of variety of foods eaten by one or more of the plurality of subjects, an indication of one or more of the plurality of subject's ability use a toilet independently, an indication of one or more of the plurality of subject's ability to bathe independently, an indication of stimulatory behaviors exhibited by one or more of the plurality of subjects, an indication of whether one or more of the plurality of subjects can be described as easy-going going with the flow, an indication of whether one or more of the plurality of subjects is anxious or easily upset by things that would not regularly upset others in otherwise similar circumstances, an indication of whether the subject follows simple directions in a home setting, or combinations thereof.

A 123^{rd} embodiment is the method of one of the 93^{rd} through the 122^{nd} embodiments, wherein the data associated with the plurality of subjects comprise the medication data, wherein the medication data comprise an indication of a medication for sleep used by one or more of the plurality of subjects, an indication of a medication for allergies used by one or more of the plurality of subjects, an indication of a medication for ASD used by one or more of the plurality of subjects, an indication of a medication for ADHD used by one or more of the plurality of subjects, an indication of a medication for anxiety used by one or more of the plurality of subjects, an indication of a medication for depression used by one or more of the plurality of subjects, an indication of a medication for a behavior or mood related condition used by one or more of the plurality of subjects, or combinations thereof.

A 124^{th} embodiment is the method of one of the 93^{rd} through the 123^{rd} embodiments, wherein the data associated with the plurality of subjects comprise the goals data, wherein the goals data comprise an indication of a goal of improved communication skills, an indication of a goal of improved diet, an indication of a goal of increased independence, an indication of a goal of improved ability to express emotions, an indication of a goal of improved social skills, an indication of a goal of improved ability to participate in family activities, an indication of a goal of decreased challenging behaviors, an indication of a goal of getting along better with parents and/or siblings, an indication of a goal of learning toilet training, an indication of a goal of learning new ways to leave non-preferred activities, an indication of a goal of doing what they are told without responding inappropriately, an indication of a goal of keeping their body and others around them safe, an indication of a goal of increased participation in general education classrooms or settings, an indication of a goal of increased flexibility and/or being less rigid, of combinations thereof.

A 125^{th} embodiment is the method of one of the 93^{rd} through the 124^{th} embodiments, wherein the data associated with the plurality of subjects further comprise diagnosis data, sleep and wake patterns data, a self-stimulatory behaviors data, restrictive and repetitive behaviors data, communication skills data, social skills data, or combinations thereof.

A 126^{th} embodiment is the method of one of the 93^{rd} through the 125^{th} embodiments, wherein the data associated with each of the plurality of subjects comprise a plurality of data features, wherein the plurality of data features comprises from about 12 to about 30 different data features.

A 127^{th} embodiment is the method of one of the 93^{rd} through the 126^{th} embodiments, wherein the data associated with each of the plurality of subjects comprise a plurality of data features, wherein the plurality of data features comprises from about 24 to about 30 different data features.

A 128^{th} embodiment is the method of one of the 105^{th} through the 107^{th} embodiments, wherein the gradient-boosted tree model comprises from about 50 decision trees to about 300 decision trees.

A 129^{th} embodiment is the method of one of the 105^{th} through the 107^{th} embodiments, wherein the gradient-boosted tree model comprises from about 50 decision trees to about 150 decision trees.

A 130^{th} embodiment is the method of one of the 109^{th} through the 129^{th} embodiments, wherein the NDDTR model hyperparameters comprise a tree depth of at least 2 and not more than 6.

A 131^{st} embodiment is the method of one of the 109^{th} through the 130^{th} embodiments, wherein the NDDTR model hyperparameters comprise a tree depth of not more than 5.

A 132^{nd} embodiment is the method of one of the 109^{th} through the 131^{st} embodiments, wherein the NDDTR model hyperparameters comprise a tree depth of not more than 3.

A 133^{rd} embodiment is the method of one of the 109^{th} through the 132^{nd} embodiments, wherein the NDDTR model hyperparameters comprise a learning rate of equal to or less than about 0.4.

A 134^{th} embodiment is the method of one of the 109^{th} through the 133^{rd} embodiments, wherein the NDDTR model hyperparameters comprise a scale positive weight of from about 0.1 to about 10.

A 135^{th} embodiment is the method of one of the 109^{th} through the 134^{th} embodiments, wherein the NDDTR model hyperparameters comprise an alpha regularization parameter of from about 0 to about 1.

A 136^{th} embodiment is the method of one of the 109^{th} through the 135^{th} embodiments, wherein the NDDTR model hyperparameters comprise a gamma regularization parameter of from about 0 to about 1.

A 137^{th} embodiment is the method of one of the 93^{rd} through the 136^{th} embodiments, wherein processing the training data associated with the plurality of subjects comprises reducing the dimensionality of the training data.

A 138^{th} embodiment is the method of the 137^{th} embodiment, wherein the training data comprise a plurality of data features; wherein the data features comprise demographic data, schooling data, family medical data, prior therapy data, observational assessment data, medication data, goals data, or combinations thereof; wherein reducing the dimensionality of the training data comprises removing (i) data features having a missing rate of greater than about 60% and/or (ii) highly correlated data features, wherein highly correlated data features have a correlation coefficient of equal to or greater than about 0.75.

A 139^{th} embodiment is the method of one of the 137^{th} through the 138^{th} embodiments, wherein reducing the dimensionality of the training data comprises performing a feature selection method selected from the group consisting of Forward Feature Selection, Backward Feature Elimination, Feature Selection based on SHAP, and combinations thereof.

A 140^{th} embodiment is the method of the 139^{th} embodiment, wherein the feature selection method further comprises (i) evaluating the area under the receiver operator characteristic curve (AUROC) of each single data feature, and (ii) removing data features that yield single feature AUROC values of equal to or less than about 0.55.

A 141^{st} embodiment is the method of the 140^{th} embodiment, wherein the feature selection method further comprises (i) evaluating the AUROC of the combined remaining data features, and (ii) iteratively training the NDDTR model by removing one data feature at a time with replacement from the data features remaining in the training dataset, wherein the NDDTR model is trained using cross-validation, and wherein feature subsets are not reshuffled between folds.

A 142^{nd} embodiment is the method of the 141^{st} embodiment further comprising eliminating one or more of the data features causing the highest increase in mean cross-validation AUROC when removed.

A 143^{rd} embodiment is the method of the 142^{nd} embodiment further comprising eliminating one or more of the data features causing a mean cross-validation AUROC of equal to or greater than 0.75 when removed.

While embodiments of the disclosure have been shown and described, modifications thereof can be made without departing from the spirit and teachings of the invention. The embodiments and examples described herein are exemplary only, and are not intended to be limiting. Many variations and modifications of the invention disclosed herein are possible and are within the scope of the invention.

Accordingly, the scope of protection is not limited by the description set out above but is only limited by the claims which follow, that scope including all equivalents of the subject matter of the claims. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, R₁, and an upper limit, Rᵤ, is disclosed, any number falling within the range is specifically disclosed. In particular, the following numbers within the range are specifically disclosed: R=R₁+k*(Rᵤ−R₁), wherein k is a variable ranging from 1 percent to 100 percent with a 1 percent increment, i.e., k is 1 percent, 2 percent, 3 percent, 4 percent, 5 percent, . . . , 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed. Also, use of the term “about” with respect to a disclosed value or other quantification should be understood to include those values proximate to the disclosed value, for example, deviating from the disclosed value by ±0.1% of the disclosed value, or ±0.5%, or ±1%, or ±2%, or ±3%, or ±4%, or ±5%, or ±6%, or ±7%, or ±8%, or ±9%, or ±10%, as contextually appropriate. Each and every claim is incorporated into the specification as an embodiment of the present invention. Thus, the claims are a further description and are in addition to the detailed description of the present invention. The disclosures of all patents, patent applications, and publications cited herein are hereby incorporated by reference.

What is claimed is:

1. A method implemented via a computing device, the method comprising:
receiving, by the computing device, data associated with a subject having a neurodevelopmental disorder (NDD), the data associated with the subject comprising demographic data, schooling data, family medical data, prior therapy data, observational assessment data, medication data, goals data, or combinations thereof, and
evaluating, by the computing device, the data associated with the subject via a neurodevelopmental disorder treatment recommendation (NDDTR) model, wherein the NDDTR model is configured to evaluate the data associated with the subject to determine a therapy recommendation, wherein the therapy recommendation comprises a standard of care.

2. The method of claim 1, wherein the neurodevelopmental disorder is autism spectrum disorder (ASD).

3. The method of claim 1, wherein the data associated with the subject comprise the demographic data, wherein the demographic data comprise age data.

4. The method of claim 1, wherein the data associated with the subject comprise the schooling data, wherein the schooling data comprise an indication of whether the subject attends school, an indication of whether the subject has been assigned a school aide, an indication of whether the subject is a part of a special education program, or combinations thereof.

5. The method of claim 1, wherein the data associated with the subject comprise the prior therapy data, wherein the prior therapy data comprise an indication of the subject having previously received occupational therapy, an indication of the subject having previously received speech therapy, an indication of duration of applied behavioral analysis (ABA) therapy previously received by the subject, an indication of amount of ABA therapy previously received by the subject, or combinations thereof.

6. The method of claim 1, wherein the data associated with the subject comprise an indication of the subject's tendency toward aggressive behavior, an indication of the subject's tendency toward stereotypy, an indication of the subject's tendency toward destructive behaviors, an indication of the consequences implemented by a caregiver of the subject responsive to negative behavior, an indication of the subject's ability to be understood, an indication of the subject's ability to understand others, an indication of variety of foods eaten by the subject, an indication of the subject's ability use a toilet independently, an indication of the subject's ability to bathe independently, an indication of stimulatory behaviors exhibited by the subject, or combinations thereof.

7. The method of claim 1, wherein the data associated with the subject comprise the goals data, wherein the goals data comprise an indication of a goal of improved communication skills, an indication of a goal of improved diet, an indication of a goal of increased independence, an indication of a goal of improved ability to express emotions, of combinations thereof.

8. The method of claim 1, wherein the data associated with the subject comprise structured data, wherein the data associated with the subject comprise a plurality of data features, and wherein the plurality of data features comprises not more than about 30 different data features.

9. The method of claim 1, wherein the NDDTR model is a machine learning model selected from the group consisting of a deep learning model, a generative adversarial network model, a computational neural network model, a recurrent neural network model, a perceptron model, a classical tree-based machine learning model, a decision tree type model, a regression type model, a classification model, a reinforcement learning model, and combinations thereof.

10. The method of claim 9, wherein the machine learning model is a gradient-boosted tree model comprising a plurality of weighted decision trees.

11. The method of claim 10, wherein the gradient-boosted tree model comprises from about 50 decision trees to about 400 decision trees, and wherein the NDDTR model has a tree depth of at least 2 and not more than 6.

12. The method of claim 10, further comprising:
identifying NDDTR model hyperparameters, wherein the NDDTR model hyperparameters comprise tree depth, number of decision trees, learning rate, scale positive weight, alpha regularization parameter, gamma regularization parameter, or combinations thereof, and
tuning the NDDTR model hyperparameters, wherein the tuning of the NDDTR model hyperparameters is effective to provide for an NDDTR model sensitivity of from about 0.75 to about 0.99.

13. The method of claim 12, wherein the gradient-boosted tree model comprises from about 50 decision trees to about 150 decision trees; wherein the NDDTR model has a tree depth of not more than 3; wherein the NDDTR model has a learning rate of equal to or less than about 0.4; wherein the NDDTR model has a scale positive weight of from about 0.1 to about 10; wherein the NDDTR model has an alpha regularization parameter of from about 0 to about 1; and wherein the NDDTR model has a gamma regularization parameter of from about 0 to about 1.

14. The method of claim 1, wherein the standard of care comprises an indication of intensity of therapy, an indication of services, or an indication of one of a comprehensive therapy or a focused therapy.

15. The method of claim 1, further comprising providing therapy to the subject based upon the therapy recommendation.

16. A computing system, the system comprising:
a computing device, the computing device comprising a processor and a non-transitory computer-readable medium, wherein the non-transitory computer-readable medium includes instructions configured to cause the processor to implement a neurodevelopmental disorder treatment recommendation (NDDTR) model, wherein the NDDTR model, when implemented via the processor, causes the computing device to:
receive data associated with a subject having a neurodevelopmental disorder (NDD), the data associated with the subject comprising demographic data, schooling data, family medical data, prior therapy data, observational assessment data, medication data, goals data, or combinations thereof, and
evaluate the data associated with the subject via a neurodevelopmental disorder treatment recommendation (NDDTR) model, wherein the NDDTR model is configured to evaluate the data associated with the subject to determine a therapy recommendation, wherein the therapy recommendation comprises a standard of care.

17. A method implemented via a computing device, the method comprising:
receiving, by the computing device, training data associated with a plurality of subjects, wherein at least a portion of the subjects are individuals characterized as having a neurodevelopmental disorder (NDD), and wherein the training data associated with each of the plurality of subjects comprise demographic data, schooling data, family medical data, prior therapy data, observational assessment data, medication data, goals data, or combinations thereof, and
processing the training data associated with the plurality of subjects to yield an NDDTR model, wherein the NDDTR model is configured to evaluate data associated with a subject to determine a therapy recommendation, wherein the therapy recommendation comprises a standard of care.

18. The method of claim 17, wherein processing the training data associated with the plurality of subjects comprises reducing the dimensionality of the training data; wherein the training data comprise a plurality of data features; wherein reducing the dimensionality of the training data comprises removing (i) data features having a missing rate of greater than about 60%, (ii) highly correlated data features, wherein highly correlated data features have a correlation coefficient of equal to or greater than about 0.75, (iii) performing a feature selection method selected from the group consisting of Forward Feature Selection, Backward Feature Elimination, Feature Selection based on SHapely Additive exPlanations (SHAP), and combinations thereof, or (iv) any combination of (i)-(iii);

19. The method of claim 18, wherein the feature selection method further comprises (1) evaluating the area under the receiver operator characteristic curve (AUROC) of each single data feature; (2) removing data features that yield single feature AUROC values of equal to or less than about 0.55; (3) evaluating the AUROC of the combined remaining data features; and (4) iteratively training the NDDTR model by removing one data feature at a time with replacement from the data features remaining in the training dataset, wherein the NDDTR model is trained using cross-validation, and wherein feature subsets are not reshuffled between folds.

20. The method of claim 19 further comprising eliminating one or more of the data features causing the highest increase in mean cross-validation AUROC when removed.
